(19) United States
(12) Patent Application Publication
(10) Pub. No.: US 2023/0340040 A1
(43) Pub. Date: Oct. 26, 2023

(54) CHIMERIC MYD88 RECEPTORS

(71) Applicant: St. Jude Children's Research Hospital, Inc., Memphis, TN (US)

(72) Inventors: Stephen GOTTSCHALK, Germantown, TN (US); Brooke PRINZING, Memphis, TN (US)

(73) Assignee: St. Jude Children's Research Hospital, Inc., Memphis, TN (US)

(21) Appl. No.: 17/918,644

(22) PCT Filed: Apr. 14, 2021
(86) PCT No.: PCT/US2021/027204

§ 371 Date: Oct. 13, 2022

Related U.S. Application Data
(60) Provisional application No. 63/059,534, filed on Jul. 31, 2020.
(60) Provisional application No. 63/009,761, filed on Apr. 14, 2020.

(51) Int. Cl. C07K 14/47; A61P 35/00; C12N 15/63

(52) U.S. Cl. CPC C07K 14/4702; A61P 35/00; C12N 15/63; C07K 2319/03

(57) ABSTRACT

The present disclosure provides chimeric MyD88 receptors. Also provided are polynucleotides encoding the chimeric MyD88 receptors, vectors comprising the polynucleotides encoding the chimeric MyD88 receptors, and cell compositions comprising the chimeric MyD88 receptors, polynucleotides and/or vectors. Pharmaceutical compositions comprising the polypeptides, polynucleotides, vectors, or cells of the present disclosure, and their uses in treating a disease in a subject are also provided.

### CROSS REFERENCE TO RELATED APPLICATIONS

This patent application claims priority to U.S. Provisional Application No. 63/059,534 filed Jul. 31, 2020 and U.S. Provisional Application No. 63/009,761, filed Apr. 14, 2020, the disclosure of both of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Apr. 14, 2021, is named 243734_000147_SL.txt and is 53,572 bytes in size.

### FIELD OF THE INVENTION

The application relates to chimeric MyD88 receptors, and their uses in immunotherapy (e.g., adoptive cell therapy) for treatment of a disease.

### BACKGROUND

Adoptive cell therapy such as chimeric antigen receptor (CAR) T cell therapy has produced extraordinary results against hematological malignancies, providing complete responses to heavily pre-treated patients with relapsed/refractory disease who have exhausted all other treatment options. Unfortunately, CAR T cells have not yet had the same success against solid tumors as well as other types of diseases. Currently, most CARs incorporate CD28 or 4-1BB as their costimulatory domain. Other costimulation approaches are yet to be explored.

Accordingly, there exists a need for improved immunotherapeutic strategies. This present invention provides a solution to address this problem.

### SUMMARY OF THE INVENTION

The present invention discloses, in various aspects, chimeric MyD88 receptors, polynucleotides encoding the chimeric MyD88 receptors, vectors comprising the polynucleotides encoding the chimeric MyD88 receptors, and host cells expressing the chimeric MyD88 receptors. Further disclosed are compositions (e.g., pharmaceutical compositions) comprising the polypeptides, polynucleotides, vectors, or host cells, and methods of using such compositions in treating a cancer in a subject.

In one aspect, provided herein is a polynucleotide encoding a chimeric MyD88 receptor comprising:
a) an extracellular domain comprising a target-binding moiety that binds to a target molecule;
b) a transmembrane domain; and
c) a cytoplasmic domain comprising a MyD88 polypeptide or a functional fragment thereof.

In some embodiments, the MyD88 polypeptide of the chimeric MyD88 receptor comprises the amino acid sequence of SEQ ID NO: 9, or an amino acid sequence having at least 80% identity thereof. In some embodiments, the nucleotide sequence encoding the MyD88 polypeptide of the chimeric MyD88 receptor comprises the nucleotide sequence of SEQ ID NO: 10, or SEQ ID NO: 26, or a nucleotide sequence having at least 80% identity thereof.

In some embodiments, the MyD88 polypeptide of the chimeric MyD88 receptor comprises the amino acid sequence of SEQ ID NO: 44, or an amino acid sequence having at least 80% identity thereof. In some embodiments, the nucleotide sequence encoding the MyD88 polypeptide of the chimeric MyD88 receptor comprises the nucleotide sequence of SEQ ID NO: 45, or a nucleotide sequence having at least 80% identity thereof.

In some embodiments, the target molecule is a molecule secreted by an immune cell that is genetically modified to express the chimeric MyD88 receptor. In some embodiments, the target molecule is interleukin 5 (IL-5), IL-6, IL-10, IL-13, IL-17, GM-CSF, RANTES (CCL5), OX40, or ICOS. In one embodiment, the target molecule is IL-13.

In some embodiments, the target molecule is expressed by a cell that is not genetically modified to express the chimeric MyD88 receptor. In some embodiments, the cell is an immune cell, cancer cell, and/or stromal cell. In some embodiments, the target molecule is programmed death-ligand 1 (PD-L1), Galectin-9, MHC class II, CD48, CD155 or CD112. In one embodiment, the target molecule is programmed death-ligand 1 (PD-L1).

In some embodiments, the target-binding moiety is an antibody or an antibody fragment. In some embodiments, the target-binding moiety is a single chain variable fragment (scFv).

In some embodiments, the target-binding moiety is derived from a cell surface receptor. In some embodiments, the target-binding moiety comprises an ectodomain of a cell surface receptor, or a functional variant or fragment thereof.

In some embodiments, the target-binding moiety is an anti-IL-13 scFv. In some embodiments, the anti-IL-13 scFv is derived from antibody hB-B13. In some embodiments, the anti-IL-13 scFv comprises the amino acid sequence of SEQ ID NO: 3, or an amino acid sequence having at least 80% identity thereof. In some embodiments, the nucleotide sequence encoding the anti-IL-13 scFv comprises the nucleotide sequence of SEQ ID NO: 4, or a nucleotide sequence having at least 80% identity thereof.

In some embodiments, the target-binding moiety is derived from PD1, TIM3, LAG3, 2B4, or TIGIT. In some embodiments, the target-binding moiety comprises an ectodomain of PD1, or a functional variant or fragment thereof. In some embodiments, the target-binding moiety derived from PD1 comprises the amino acid sequence of SEQ ID NO: 21, or SEQ ID NO: 30, or an amino acid sequence having at least 80% identity thereof. In some embodiments, the nucleotide sequence encoding the target-binding moiety derived from PD1 comprises the nucleotide sequence of SEQ ID NO: 22, or SEQ ID NO: 31, or a nucleotide sequence having at least 80% identity thereof.

In some embodiments, the transmembrane domain is derived from CD28, CD8, CD4, CD3ζ, CD40, CD134 (OX-40), CD19, or CD7. In some embodiments, the transmembrane domain is derived from CD28. In some embodiments, the transmembrane domain of the chimeric MyD88 receptor comprises the amino acid sequence of SEQ ID NO: 7, or an amino acid sequence having at least 80% sequence identity thereof. In some embodiments, the nucleotide sequence encoding the transmembrane domain of the chimeric MyD88 receptor comprises the nucleotide sequence of SEQ ID NO: 8, or SEQ ID NO: 25, or a nucleotide sequence having at least 80% sequence identity thereof.

In some embodiments, the extracellular domain further comprises a hinge domain between the target-binding moiety and the transmembrane domain. In some embodiments, the hinge domain is derived from IgG1, IgG4, CD28, or CD8. In some embodiments, the hinge domain is derived from IgG1. In some embodiments, the hinge domain comprises the amino acid sequence of SEQ ID NO: 5, or an amino acid sequence having at least 80% sequence identity thereof. In some embodiments, the nucleotide sequence encoding the hinge domain comprises the nucleotide sequence of SEQ ID NO: 6, or a nucleotide sequence having at least 80% sequence identity thereof. In some embodiments, the hinge domain is derived from CD28. In some embodiments, the hinge domain comprises the amino acid sequence of SEQ ID NO: 23, or an amino acid sequence having at least 80% sequence identity thereof. In some embodiments, the nucleotide sequence encoding the hinge domain comprises the nucleotide sequence of SEQ ID NO: 24, or a nucleotide sequence having at least 80% sequence identity thereof.

In some embodiments, the extracellular domain further comprises a leader sequence. In some embodiments, the leader sequence is derived from CD8a, PD1, or human immunoglobulin heavy chain variable region. In some embodiments, the leader sequence comprises the amino acid sequence of SEQ ID NO: 1, or an amino acid sequence having at least 80% sequence identity thereof. In some embodiments, the nucleotide sequence encoding the leader sequence comprises the nucleotide sequence of SEQ ID NO: 2, or a nucleotide sequence having at least 80% sequence identity thereof. In some embodiments, the leader sequence comprises the amino acid sequence of SEQ ID NO: 19, or an amino acid sequence having at least 80% sequence identity thereof. In some embodiments, the nucleotide sequence encoding the leader sequence comprises the nucleotide sequence of SEQ ID NO: 20, or SEQ ID NO: 29, or a nucleotide sequence having at least 80% sequence identity thereof.

In some embodiments, the chimeric MyD88 receptor comprises the amino acid sequence of SEQ ID NO: 15, or an amino acid sequence having at least 80% sequence identity thereof. In some embodiments, the nucleotide sequence encoding the chimeric MyD88 receptor comprises the nucleotide sequence of SEQ ID NO: 16, or a nucleotide sequence having at least 80% sequence identity thereof.

In some embodiments, the chimeric MyD88 receptor comprises the amino acid sequence of SEQ ID NO: 27, or an amino acid sequence having at least 80% sequence identity thereof. In some embodiments, the nucleotide sequence encoding the chimeric MyD88 receptor comprises the nucleotide sequence of SEQ ID NO: 28, or a nucleotide sequence having at least 80% sequence identity thereof.

In some embodiments, the chimeric MyD88 receptor comprises the amino acid sequence of SEQ ID NO: 32, or an amino acid sequence having at least 80% sequence identity thereof. In some embodiments, the nucleotide sequence encoding the chimeric MyD88 receptor comprises the nucleotide sequence of SEQ ID NO: 33, or a nucleotide sequence having at least 80% sequence identity thereof.

In some embodiments, the polynucleotide further encodes at least one additional polypeptide. In some embodiments, the at least one polypeptide is a transduced host cell selection marker, an in vivo tracking marker, a cytokine, or a safety switch gene. In some embodiments, the transduced host cell selection marker is a truncated CD19 (tCD19) polypeptide. In some embodiments, the tCD19 comprises the amino acid sequence SEQ ID NO: 13, or an amino acid sequence having at least 80% sequence identity thereof. In some embodiments, the nucleotide sequence encoding the tCD19 comprises the nucleotide sequence SEQ ID NO: 14, or an amino acid sequence having at least 80% sequence identity thereof.

In some embodiments, the sequence encoding the chimeric MyD88 receptor is operably linked to the sequence encoding at least an additional polypeptide sequence via a sequence encoding a self-cleaving peptide and/or an internal ribosomal entry site (IRES). In some embodiments, the self-cleaving peptide is a 2A peptide, e.g., T2A, P2A, E2A, or F2A peptide. In some embodiments, the 2A peptide is a T2A peptide. In some embodiments, the T2A peptide comprises the amino acid sequence SEQ ID NO: 11, or an amino acid sequence having at least 80% sequence identity thereof. In some embodiments, the sequence encoding the T2A peptide comprises the nucleotide sequence SEQ ID NO: 12, or a nucleotide sequence having at least 80% sequence identity thereof.

In some embodiments, the polynucleotide encoding a chimeric MyD88 receptor encodes the amino acid sequence of SEQ ID NO: 17, or an amino acid sequence having at least 80% sequence identity thereof. In some embodiments, the polynucleotide encoding a chimeric MyD88 receptor comprises the nucleotide sequence of SEQ ID NO: 18, or a nucleotide sequence having at least 80% sequence identity thereof.

In some embodiments, the polynucleotide encoding a chimeric MyD88 receptor is a DNA molecule.

In some embodiments, the polynucleotide encoding a chimeric MyD88 receptor is an RNA molecule.

In another aspect, provided herein is a chimeric MyD88 receptor encoded by the polynucleotide of any one of the embodiments described above.

In another aspect, provided herein is a recombinant vector comprising the polynucleotide of any one of the embodiments described above. In some embodiments, the vector is a viral vector. In some embodiments, the viral vector is a retroviral vector, a lentiviral vector, an adenoviral vector, an adeno-associated virus vector, an alphaviral vector, a herpes virus vector, a baculoviral vector, or a vaccinia virus vector. In some embodiments, the viral vector is a retroviral vector. In some embodiments, the vector is a non-viral vector. In some embodiments, the non-viral vector is a minicircle plasmid, a Sleeping Beauty transposon, a piggyBac transposon, or a single or double stranded DNA molecule that is used as a template for homology directed repair (HDR) based gene editing.

In another aspect, provided herein is an isolated host cell comprising the polynucleotide of any one of the embodiments described above or the recombinant vector of any one of the embodiments described above.

In another aspect, provided herein is an isolated host cell comprising a chimeric MyD88 receptor encoded by the polynucleotide of any one of the embodiments described above. In some embodiments, the host cell is an immune cell. In some embodiments, the host cell is a T cell, a natural killer (NK) cell, a mesenchymal stem cell (MSC), or a macrophage. In some embodiments, the host cell is a T cell. In some embodiments, the host cell is an αβ T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (T_{SCM}), a naive T-cell, an effector T-cell, a T-helper cell, or a regulatory T-cell (Treg).

In some embodiments, the host cell further expresses a molecule that directs the host cell to a target cell. In some embodiments, the molecule that directs the host cell to a target cell is a chimeric antigen receptor (CAR), a T cell antigen coupler (TAC), an αβ TCR, or a bispecific antibody. In some embodiments, the bispecific antibody is a bispecific T-cell engager (BiTE), or a dual-affinity re-targeting (DART) antibody.

In some embodiments, the molecule that redirects the immune cell to target cells comprises an antigen-binding domain that specifically binds to an antigen. In some embodiments, the antigen is a tumor-associated antigen (TAA), an antigen expressed in the tumor stroma, an antigen expressed on endothelial cell, a virus-associated antigen, or an antigen expressed on immune and/or stem cell.

In some embodiments, the antigen is selected from i) tumor-associated antigens (TAAs) including, but not limited to, human epidermal growth factor receptor 2 (HER2), Eph receptor A2 (EphA2), B7-H3 (CD276), interleukin 13 receptor alpha 2 (IL13Rα2), cell surface GRP78, CD19, CD123; ii) antigens expressed in the tumor stroma including, but not limited to, oncofetal splice variants of fibronectin and tenascin C, fibroblast activating protein (FAP), iii) antigens expressed on endothelial cells including, but not limited to, VEGF receptors, tumor endothelial markers (TEMs), iv) virus-associated antigens including, but not limited to, HBsAg, and v) antigens expressed on immune and/or stem cells to deplete these cells including, but not limited to, CD45RA, c-kit.

In some embodiments, the host cell has further been modified such that the expression and/or function of one or more gene(s) or gene product(s) in said host cell is reduced or eliminated. In some embodiments, the one or more gene(s) comprises IRAK3.

In some embodiments, the host cell has been activated and/or expanded ex vivo.

In some embodiments, the host cell is an allogeneic cell. In some embodiments, the host cell is an autologous cell.

In some embodiments, the host cell is isolated from a subject having a cancer. In some embodiments, the cancer is a HER2-positive or EphA2-positive cancer.

In some embodiments, the host cell is derived from a blood, marrow, tissue, or a tumor sample.

In another aspect, provided herein is a pharmaceutical composition comprising the host cell of any one of the embodiments described above and a pharmaceutically acceptable carrier and/or excipient.

In another aspect, provided herein is a method of generating the isolated host cell of any one of the embodiments described above, said method comprising genetically modifying the host cell with the polynucleotide of any one of the embodiments described above or the recombinant vector of any one of the embodiments described above. In some embodiments, the method further comprises genetically modifying the host cell to expresses molecule that redirects the immune cell to target cells. In some embodiments, the genetic modifying step is conducted via viral gene delivery. In some embodiments, the genetic modifying step is conducted via non-viral gene delivery. In some embodiments, the method further comprises modifying one or more gene(s) or gene product(s) in the host cell such that the expression and/or function of one or more gene(s) or gene product(s) in said host cell is reduced or eliminated. In some embodiments, the one or more gene(s) comprises IRAK3. In some embodiments, the modifying step comprises disrupting the one or more gene(s) with a site-specific nuclease; or silencing an mRNA expressed from the one or more gene(s) with an RNA interference (RNAi) molecule or an antisense oligonucleotide; or inhibiting a protein expressed from the one or more gene(s) with one or more of a small molecule inhibitor, a peptide, an antibody or antibody fragment, and an aptamer. In some embodiments, the genetic modification is conducted ex vivo. In some embodiments, the method further comprises activation and/or expansion of the host cell ex vivo before, after and/or during said genetic modification.

In another aspect, provided herein is a method for killing a target cell, said method comprising contacting said cell with the host cell(s) of any one of the embodiments described above or the pharmaceutical composition described above.

In another aspect, provided herein is a method for treating a disease in a subject in need thereof, said method comprising administering to the subject a therapeutically effective amount of the host cell(s) of any one of the embodiments described above or the pharmaceutical composition described above.

In some embodiments, the method for treating a disease comprises:
a) isolating T cells, NK cells, or macrophages from the subject;
b) genetically modifying said T cells, NK cells, or macrophages ex vivo with the polynucleotide of any one of the embodiments described above or the vector of any one of the embodiments described above;
c) optionally, genetically modifying said T cells, NK cells, or macrophages ex vivo to express a molecule that redirects said cells to a target cell and/or modifying the IRAK3 gene or gene product(s) thereof in said cells such that the expression and/or function of IRAK3 or gene product(s) thereof in said cells is reduced or eliminated;
d) optionally, expanding and/or activating said T cells, NK cells, or macrophages before, after or during step (b) or (c); and
e) introducing the genetically modified T cells, NK cells, or macrophages into the subject.

In some embodiments of the therapeutic method described above, the disease is cancer, an infectious disease, or an autoimmune disease. In some embodiments, the target cell is a cancer cell, a pathogen, or an auto-reactive immune cell.

In various embodiments described above, the subject is human.

These and other aspects of the present invention will be apparent to those of ordinary skill in the art in the following description, claims and drawings.

### BRIEF DESCRIPTION OF THE DRAWINGS

FIGS. 1A-1B show schematics of chimeric MyD88 receptors to provide costimulation separate from the CD3ζ-containing CAR. In FIG. 1A, when the CAR binds its cognate tumor-associated antigen (TAA), activation of transcription factors such as NFAT (nuclear factor of activated T cells) results in the production of cytokines, including IL-13. IL-13 can then bind to the anti-IL13(α13)-MyD88 receptor to provide costimulatory signaling. In FIG. 1B, co-expressing a CAR with a PD1-MyD88 receptor allows for CD3ζ signaling to be provided by the CAR binding its cognate TAA while MyD88 costimulation results from the PD1-MyD88 receptor binding PDL1 on the tumor cell surface or other cells within the tumor microenvironment.

FIGS. 2A-2B show schematics of chimeric MyD88 receptors. FIG. 2A is a schematic of α13-MyD88 receptor. SP: signal peptide; TM: transmembrane; t: truncated. FIG. 2B shows schematics of PD1-MyD88 receptors and corresponding dissociation constant (K_{D}). The mutations shown were incorporated into the PD1 domain of the PD1-MyD88 receptor to increase the binding affinity to PDL (chimeric PD1 high affinity-MyD88 (PD1H-MyD88) receptor).

FIGS. 3A-3C demonstrate that chimeric MyD88 receptors can be expressed on T cells and bind their intended targets. Flow cytometry was used to determine chimeric MyD88 receptor expression on the T cell surface. Nontransduced (NT) T cells served as a control. In FIG. 3A, T cells transduced with the α13-MyD88 receptor were stained with a CD19 antibody to detect transduced cells. In FIG. 3B, expression of chimeric PD1-MyD88 receptors was determined using a PD1 antibody. In FIG. 3C, T cells were incubated with recombinant human IL-13-FC or PDL1-FC followed by an FC antibody to determine if the chimeric MyD88 receptors bind their intended targets.

FIGS. 4A-4B demonstrate that chimeric PD1-MyD88 receptors increase expression of Bcl-xL and Ki67 after stimulation in the presence of PDL1. CAR T cells transduced with a HER2 CAR+/−PD1-MyD88 or PD1H-MyD88 were stimulated with recombinant human HER2 and PDL1 for 24 hours before being permeabilized and stained for the anti-apoptotic protein Bcl-xL (FIG. 4A) and the marker of proliferation Ki67 (FIG. 4B).

FIG. 5A-5B demonstrate that activated T cells produce IL-13 and induce expression of PDL1 on tumor cells. In FIG. 5A, T cells were incubated with U373 glioma cells at a 2:1 effector:target ratio. After 24 hours, the supernatant was collected and the production of IL-13 was assessed using a milliplex cytokine assay. In FIG. 5B, flow cytometry was used to assess the expression of PDL1 on the surface of U373 and LM7 tumor cells at baseline and after 24 hours exposure to supernatant from T cells that had been activated with αCD3/uCD28 for 24 hours.

FIGS. 6A-6B demonstrate that chimeric MyD88 receptors enhance T cell proliferation without abrogating CAR cytotoxicity. In FIG. 6A, tumor cells were incubated with T cells at varying effector:target (E:T) ratios for 24 hours before the number of live tumor cells was quantified using an MTS assay. In FIG. 6B, T cells were co-cultured with tumor cells at a 2:1 E:T ratio for seven days and re-stimulated with fresh tumor cells on a weekly basis for as long as they continued to expand. Fold T cell expansion over time is shown.

FIGS. 7A-7C show that chimeric MyD88 receptors improve anti-tumor activity of HER2 CAR T cells in vivo. NSG mice were injected i.p. with 1×10⁶ LM7-ffluc on day 0. On day 7, mice were injected with 1×10⁵ CAR T cells. Bioluminescence imaging was used to track tumor burden over time. FIG. 7A shows total flux; FIG. 7B shows representative images; and FIG. 7C shows survival curve of mice treated with HER2.CD28.ζ CAR T cells +/−α13-MyD88 or PD1H-MyD88.

FIGS. 8A-8B show that MyD88.CD40 CAR T cells express high levels of ZCH12A and IRAK3 before and after stimulation. In FIG. 8A, EphA2 Delta, CD28, 4-1BB and MC CAR T cells were taken from culture (Base) or stimulated with LM7 tumor cells (Stim) for 24 hours before being sorted into CD4+ and CD8+ T cells are shown (n=3, 2-way ANOVA with Turkey's test for multiple comparisons). For each bar graph, bars corresponding to data generated using EphA2 Delta, CD28, 4-1BB and MC CAR T cells are shown in order of appearance (from left to right) for the Base and Stim conditions. In FIG. 8B, EphA2 CAR T cells were taken directly from culture or stimulated with recombinant human EphA2 protein for 24 hours before being lysed and used for Western Blot. The membrane was probed for IRAK3 and GAPDH as a loading control.

FIG. 9 shows the protein sequence of anti-IL13-CD28TM-MyD88-2A-tCD19 (α13-MyD88). FIG. 9 discloses SEQ ID NO: 17.

FIG. 10 shows the nucleotide sequence encoding the anti-IL13-CD28TM-MyD88-2A-tCD19 (α13-MyD88). FIG. 10 discloses SEQ ID NO: 18.

FIG. 11 shows the protein sequence of PD1-CD28TM-MyD88 (PD1-MyD88). FIG. 11 discloses SEQ ID NO: 27.

FIG. 12 shows the nucleotide sequence encoding the PD1-CD28TM-MyD88 (PD1-MyD88). FIG. 12 discloses SEQ ID NO: 28.

FIG. 13 shows the protein sequence of high-affinity PD1-CD28TM-MyD88 (PD1H-MyD88). FIG. 13 discloses SEQ ID NO: 32.

FIG. 14 shows the nucleotide sequence encoding high-affinity PD1-CD28TM-MyD88 (PD1H-MyD88). FIG. 14 discloses SEQ ID NO: 33.

### DETAILED DESCRIPTION

### Definitions

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant as T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs” refers to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+ T cells.

The terms “natural killer cell” and “NK cell” are used interchangeable and used synonymously herein. As used herein, NK cell refers to a differentiated lymphocyte with a CD 16+CD56+ and/or CD57+ TCR-phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “chimeric antigen receptor” or “CAR” as used herein is defined as a cell-surface receptor comprising an extracellular target-binding domain, a transmembrane domain and a cytoplasmic domain, comprising a signaling domain and optionally at least one costimulatory signaling domain, all in a combination that is not naturally found together on a single protein. This particularly includes receptors wherein the extracellular domain and the cytoplasmic domain are not naturally found together on a single receptor protein. The chimeric antigen receptors described herein may be used with lymphocytes such as T cells and natural killer (NK) cells.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′)2, and Fv fragments; polypeptides derived from T Cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. In addition, peptides can be used (e.g., TiE peptide, binding peptides for GRP78 or chlorotoxin). Combinatorial libraries could also be used to identify peptides binding with high affinity to the therapeutic target.

Terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12.

Host cells of the present disclosure include T cells and natural killer cells that contain the DNA or RNA sequences encoding the chimeric MyD88 receptor and express the chimeric MyD88 receptor on the cell surface. Host cells may be used for enhancing T cell activity, natural killer cell activity, treatment of cancer, and treatment of autoimmune disease.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Costimulatory signals can amplify the magnitude of the primary signals and suppress cell death following initial stimulation resulting in a more durable activation state and thus a higher cytotoxic capacity. A “costimulatory signal” refers to a signal, which in combination with a primary signal, such as TCR/CD3 ligation, leads to T cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular, or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric MyD88 receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” or “genetically engineered” refers to the addition of extra genetic material in the form of DNA or RNA into a cell.

As used herein, the term “variant” in the context of proteins or polypeptides (e.g., chimeric MyD88 receptor constructs or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a variant of, (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a variant of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a variant of, (d) a polypeptide encoded by nucleic acids can hybridize under high, moderate or typical stringency hybridization conditions to nucleic acids encoding the polypeptide it is a variant of, (e) a polypeptide encoded by a nucleotide sequence that can hybridize under high, moderate or typical stringency hybridization conditions to a nucleotide sequence encoding a fragment of the polypeptide, it is a variant of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a variant of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The term “functional fragment” as used herein refers to a fragment of the polypeptide or protein, or a polynucleotide encoding the polypeptide or protein, that retains at least one function of the full-length polypeptide or protein. A functional fragment may comprise an amino acid sequence of at least 5 contiguous amino acid residues, at least 6 contiguous amino acid residues, at least 7 contiguous amino acid residues, at least 8 contiguous amino acid residues, at least 9 contiguous amino acid residues, at least 10 contiguous amino acid residues, at least 11 contiguous amino acid residues, at least 12 contiguous amino acid residues, at least 13 contiguous amino acid residues, at least 14 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of the full-length polypeptide or protein. The functional fragment of a polypeptide or protein may retain one, two, three, four, five, or more functions of the full-length protein or polypeptide. For example, a functional fragment of a MyD88 polypeptide may retain the ability to carry out MyD88-mediated signaling. In one embodiment, the functional fragment of a MyD88 polypeptide comprises the MyD88 endodomain.

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E.W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric MyD88 Receptor

In one aspect, provided herein is a polynucleotide encoding a chimeric MyD88 receptor comprising:
a) an extracellular domain comprising a target-binding moiety that binds to a target molecule;
b) a transmembrane domain; and
c) a cytoplasmic domain comprising a MyD88 polypeptide or a functional fragment thereof.

In another aspect, provided herein is a chimeric MyD88 receptor comprising:
a) an extracellular domain comprising a target-binding moiety that binds to a target molecule;
b) a transmembrane domain; and
c) a cytoplasmic domain comprising a MyD88 polypeptide or a functional fragment thereof.

In some embodiments, the MyD88 polypeptide comprises the amino acid sequence of SEQ ID NO: 9, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In some embodiments, the nucleotide sequence encoding the MyD88 polypeptide comprises the nucleotide sequence encoding the amino acid sequence of SEQ ID NO: 9, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In some embodiments, the nucleotide sequence encoding the MyD88 polypeptide comprises the nucleotide sequence of SEQ ID NO: 10, or SEQ ID NO: 26, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In some embodiments, the MyD88 polypeptide comprises the amino acid sequence of SEQ ID NO: 44, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In some embodiments, the nucleotide sequence encoding the MyD88 polypeptide comprises the nucleotide sequence encoding the amino acid sequence of SEQ ID NO: 44, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In some embodiments, the nucleotide sequence encoding the MyD88 polypeptide comprises the nucleotide sequence of SEQ ID NO: 45, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

### Leader Sequence

In certain embodiments, the chimeric MyD88 receptor of the present disclosure comprises a leader sequence. The leader sequence may be positioned amino-terminal to the extracellular domain. The leader sequence may be optionally cleaved from the target-binding moiety during cellular processing and localization of the chimeric MyD88 receptor to the cellular membrane.

In some embodiments, the leader sequence is derived from CD8 (e.g., CD8U), PD1, or human immunoglobulin heavy chain variable region.

In one embodiment, the leader sequence comprises the amino acid sequence of SEQ ID NO: 1, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the leader sequence comprises the nucleotide sequence encoding the amino acid sequence of SEQ ID NO: 1, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the leader sequence comprises the nucleotide sequence of SEQ ID NO: 2, or a nucleotide sequence having at least 80% sequence identity thereof.

In one embodiment, the leader sequence comprises the amino acid sequence of SEQ ID NO: 19, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the leader sequence comprises the nucleotide sequence encoding the amino acid sequence of SEQ ID NO: 19, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the leader sequence comprises the nucleotide sequence of SEQ ID NO: 20, or SEQ ID NO: 29, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

### Extracellular Domain

In certain embodiments, chimeric MyD88 receptors of the present disclosure comprise an extracellular domain, wherein the extracellular domain comprises an target-binding moiety that binds to a target molecule.

In various embodiments, the target-binding moiety is an antibody or an antibody fragment. Target-binding moieties may comprise antibodies and/or antibody fragments such as monoclonal antibodies, multispecific antibodies, chimeric antibodies, single-chain Fvs (scFv), single chain antibodies, Fab fragments, F(ab′) fragments, disulfide-linked Fvs (sdFv), intrabodies, minibodies, single domain antibody variable domains, nanobodies (VHHs), diabodies and anti-idiotypic (anti-Id) antibodies (including, e.g., anti-Id antibodies to antigen specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains. In some embodiments, the target-binding moiety is a single chain variable fragment (scFv).

In some embodiments, the target-binding moiety is derived from a cell surface receptor. For example, the target-binding moiety may comprise an ectodomain of a cell surface receptor, or a functional variant or fragment thereof. Exemplary cell surface receptors that are suitable for engineering the chimeric MyD88 receptor include, but are not limited to, PD1, TIM3, LAG3, 2B4, or TIGIT.

In some embodiments, the target molecule is a molecule secreted by a host cell that is genetically modified to express the chimeric MyD88 receptor. In some embodiments, the host cell is an immune cell. In some embodiments, secretion of the molecule occurs upon activation of the immune cell. In some embodiments, the target molecule is interleukin 5 (IL-5), IL-6, IL-13, IL-17, GM-CSF, RANTES (CCL5), OX40, or ICOS.

In other embodiments, the target molecule is expressed by a cell that is not genetically modified to express the chimeric MyD88 receptor. Such cells may include but are not limited to, immune cells, cancer cells, and/or stromal cells. In some embodiments, the target molecule is programmed death-ligand 1 (PD-L1), Galectin-9, MHC class II, CD48, CD155 or CD112.

In a particular embodiment, the target molecule is IL-13.

In some embodiments, the target-binding moiety is an anti-IL-13 scFv. In some embodiments, the anti-IL-13 scFv is derived from antibody hB-B13. In a particular embodiment, the anti-IL-13 scFv comprises the amino acid sequence SEQ ID NO: 3, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In a particular embodiment, the nucleotide sequence encoding the anti-IL-13 scFv comprises the nucleotide sequence encoding the amino acid sequence SEQ ID NO: 3, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In a particular embodiment, the nucleotide sequence encoding the anti-IL-13 scFv comprises the nucleotide sequence SEQ ID NO: 4, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In a particular embodiment, the target molecule is PD-L1.

In some embodiments, the target-binding moiety comprises an ectodomain of PD1, or a functional variant or fragment thereof. In a particular, the target-binding moiety derived from PD1 comprises the amino acid sequence of SEQ ID NO: 21, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In a particular, the nucleotide sequence encoding the target-binding moiety derived from PD1 comprises the nucleotide sequence encoding the amino acid sequence of SEQ ID NO: 21, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In a particular, the nucleotide sequence encoding the target-binding moiety derived from PD1 comprises the nucleotide sequence of SEQ ID NO: 22, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

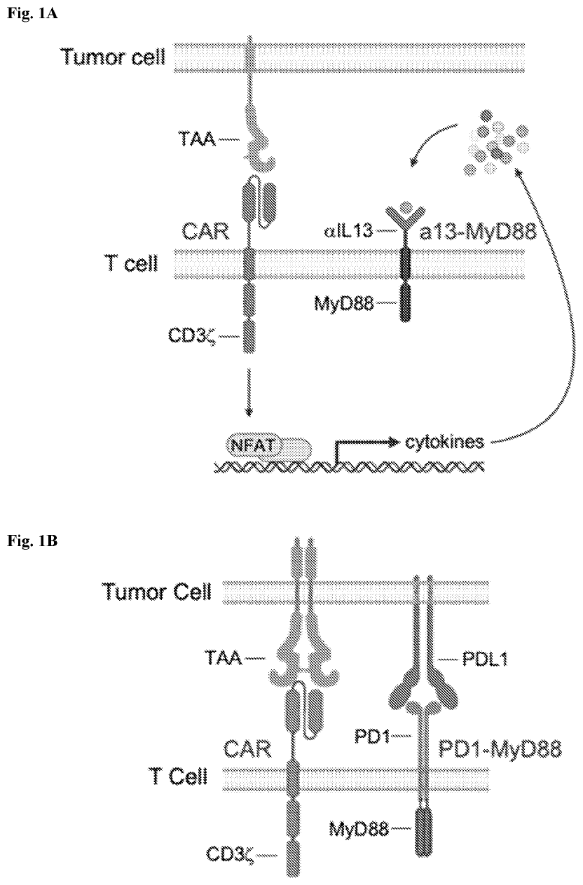

In some embodiments, the target-binding moiety comprises a functional variant of the ectodomain of PD1. The variant of ectodomain of PD1 may contain mutations that improve the affinity of the PD1 ectodomain to its ligand PD-L1. In some embodiments, the variant of ectodomain of PD1 may be a variant described in FIG. 1C of Maute et al., Proc Natl Acad Sci USA. 2015 Nov. 24; 112(47): E6506-E6514, which is incorporated herein by reference in its entirety. In some embodiments, the variant of the ectodomain of PD1 comprises one or more mutations V39H, L40V, N41V, Y43H, M45E, N49G, K53T, L97V, A100V, and/or A107I with respect to the wild-type sequence of the ectodomain of PD1. In one embodiment, the variant of the ectodomain of PD1 comprises mutations V39H, L40V, N41V, Y43H, M45E, N49G, K53T, L97V, A100V, and A107I with respect to the wild-type sequence of the ectodomain of PD1.

In a particular embodiment, the target-binding moiety derived from PD1 comprises the amino acid sequence of SEQ ID NO: 30, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In a particular embodiment, the nucleotide sequence encoding the target-binding moiety derived from PD1 comprises the nucleotide sequence encoding the amino acid sequence of SEQ ID NO: 30, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In a particular embodiment, the nucleotide sequence encoding the target-binding moiety derived from PD1 comprises the nucleotide sequence of SEQ ID NO: 31, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

### Linker Region and Hinge Domain

In certain embodiments, the chimeric MyD88 receptor further comprises a linker region between the extracellular domain and the transmembrane domain, wherein the target-binding moiety, linker, and the transmembrane domain are in frame with each other.

The term “linker region” as used herein generally means any oligo- or polypeptide that functions to link the antigen-binding moiety to the transmembrane domain. A linker region can be used to provide more flexibility and accessibility for the antigen-binding moiety. A linker region may comprise up to 300 amino acids, preferably 10 to 100 amino acids and most preferably 25 to 50 amino acids. A linker region may be derived from all or part of naturally occurring molecules, such as from all or part of the extracellular region of CD8, CD4 or CD28, or from all or part of an antibody constant region. Alternatively, the linker region may be a synthetic sequence that corresponds to a naturally occurring linker region sequence, or may be an entirely synthetic linker region sequence. Non-limiting examples of linker regions which may be used in accordance to the invention include a part of human CD8a chain, partial extracellular domain of CD28, FcγRllla receptor, IgG, IgM, IgA, IgD, IgE, an Ig hinge, or functional fragment thereof. In some embodiments, additional linking amino acids are added to the linker region to ensure that the antigen-binding moiety is an optimal distance from the transmembrane domain. In some embodiments, when the linker is derived from an Ig, the linker may be mutated to prevent Fc receptor binding.

In some embodiments, the linker domain comprises a hinge domain. The hinge domain may be derived from CD8a, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In some embodiments, the hinge domain is derived from IgG1, IgG4, CD28, or CD8.

In one embodiment, the hinge domain is derived from IgG1. In one embodiment, the hinge domain comprises the amino acid sequence of SEQ ID NO: 5, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the hinge domain comprises the nucleotide sequence encoding the amino acid sequence of SEQ ID NO: 5, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the hinge domain comprises the nucleotide sequence of SEQ ID NO: 6, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In one embodiment, the hinge domain is derived from CD28. In one embodiment, the hinge domain comprises the amino acid sequence of SEQ ID NO: 23, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the hinge domain comprises the nucleotide sequence encoding the amino acid sequence of SEQ ID NO: 23, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the hinge domain comprises the nucleotide sequence of SEQ ID NO: 24, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In some embodiments, in addition to the hinge domain, the linker region comprises additional linker amino acids to allow for extra flexibility and/or accessibility.

### Transmembrane Domain

In certain aspects, chimeric MyD88 receptors of the present disclosure comprise a transmembrane domain, fused in frame between the extracellular domain and the cytoplasmic domain.

The transmembrane domain may be derived from the protein contributing to the extracellular target-binding domain, the protein contributing the signaling or co-signaling domain, or by a totally different protein. In some instances, the transmembrane domain can be selected or modified by amino acid substitution, deletions, or insertions to minimize interactions with other members of the chimeric MyD88 receptor. In some instances, the transmembrane domain can be selected or modified by amino acid substitution, deletions, or insertions to avoid-binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this invention may be derived from (i.e. comprise at least the transmembrane region(s) of) the α, β or ζ chain of the T-cell receptor, CD28, CD3 epsilon, CD45, CD4, CD5, CD8, CD9, CD16, CD22, CD33, CD37, CD40, CD64, CD80, CD86, CD134, CD137, or CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, the transmembrane domain is derived from CD28, CD8, CD4, CD3ζ, CD40, CD134 (OX-40), CD19, or CD7.

In some embodiments, the transmembrane domain is derived from CD28. In one embodiment, the transmembrane domain of the chimeric MyD88 receptor comprises the amino acid sequence of SEQ ID NO: 7, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the transmembrane domain of the chimeric MyD88 receptor comprises the nucleotide sequence encoding the amino acid sequence of SEQ ID NO: 7, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the transmembrane domain of the chimeric MyD88 receptor comprises the nucleotide sequence of SEQ ID NO: 8, or SEQ ID NO: 25, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In some embodiments, the chimeric MyD88 receptor may comprise additional linker amino acids between the transmembrane domain and the cytoplasmic domain to allow for flexibility and/or accessibility.

### Accessory Genes

Chimeric MyD88 receptors of the present disclosure may further comprise an accessory gene that encodes an accessory peptide. Examples of accessory genes can include a transduced host cell selection marker, an in vivo tracking marker, a cytokine, a suicide gene, a safety switch gene, or some other functional gene. In certain embodiments, the functional accessory gene can increase the safety of the chimeric MyD88 receptor. In certain embodiments, the chimeric MyD88 receptor comprises at least one accessory gene. In certain embodiments, the chimeric MyD88 receptor comprises one accessory gene. In other embodiments, the chimeric MyD88 receptor comprises two accessory genes. In yet another embodiment, the chimeric MyD88 receptor comprises three accessory genes.

Non-limiting examples of additional classes of accessory genes that can be introduced into the chimeric MyD88 receptor containing host cells, include (a) chimeric antigen receptors (CAR), (b) T cell antigen couplers (TAC), (c) up TCRs, (d) antibodies, including fragments thereof and bispecific antibodies (e.g., but not limited to, bispecific T-cell engagers (BiTEs), or a dual-affinity re-targeting (DART) antibody), (e) secretable cytokines (e.g., but not limited to, IL-7, IL-12, IL-15, IL-18), (f) membrane bound cytokines (e.g., but not limited to, IL-15), (g) chimeric cytokine receptors (e.g., but not limited to, IL-2/IL-7, IL-4/IL-7), (h) constitutive active cytokine receptors (e.g., but not limited to, C7R), (i) dominant negative receptors (DNR; e.g., but not limited to TGFRII DNR), or (j) ligands of costimulatory molecules (e.g., but not limited to, CD80, 4-1BBL).

For example, the chimeric MyD88 receptor construct may comprise an accessory gene which is a truncated CD19 (tCD19). The tCD19 can be used as a tag. Expression of tCD19 may also help determine transduction efficiency. In one embodiment, the tCD19 comprises the amino acid sequence SEQ ID NO: 13, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the tCD19 comprises the nucleotide sequence encoding the amino acid sequence SEQ ID NO: 13, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the tCD19 comprises the nucleotide sequence SEQ ID NO: 14, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof.

In certain embodiments, the functional accessory gene can be a suicide gene. A suicide gene is a recombinant gene that will cause the host cell that the gene is expressed in to undergo programmed cell death or antibody mediated clearance at a desired time. Suicide genes can function to increase the safety of the chimeric MyD88 receptor. In another embodiment, the accessory gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105(11): 4247-4254; US Publ. No. 2011/0286980, each of which are incorporated herein by reference in their entirety for all purposes)).

In some embodiments, the sequence encoding the chimeric MyD88 receptor is operably linked to the sequence encoding at least an additional polypeptide sequence via a sequence encoding a self-cleaving peptide and/or an internal ribosomal entry site (IRES).

Non-limiting examples of self-cleaving peptide sequences includes Thoseaasigna virus 2A (T2A; EGRGSLLTCGDVEENPGP, SEQ ID NO: 11 or GSGEGRGSLLTCGDVEENPGP, SEQ ID NO: 34); the foot and mouth disease virus (FMDV) 2A sequence (F2A; GSGSRVTELLYRMKRAETYCPRPLLAIHPTEARHKQKIVAPVKQLLNFDLLKLAGDVES NPGP, SEQ ID NO: 35), Sponge (Amphimedon queenslandica) 2A sequence (LLCFLLLLLSGDVELNPGP, SEQ ID NO: 36; or HHFMFLLLLLAGDIELNPGP, SEQ ID NO: 37); acorn worm 2A sequence (Saccoglossus kowalevskii) (WFLVLLSFILSGDIEVNPGP, SEQ ID NO: 38); amphioxus (Branchiostoma floridae) 2A sequence (KNCAMYMLLLSGDVETNPGP, SEQ ID NO: 39; or MVISQLMLKLAGDVEENPGP, SEQ ID NO: 40); porcine teschovirus-1 2A sequence (P2A; GSGATNFSLLKQAGDVEENPGP, SEQ ID NO: 41); and equine rhinitis A virus 2A sequence (E2A; GSGQCTNYALLKLAGDVESNPGP, SEQ ID NO: 42). In some embodiments, the separation sequence is a naturally occurring or synthetic sequence. In certain embodiments, the separation sequence includes the 2A consensus sequence D-X-E-X-NPGP (SEQ ID NO: 43), in which X is any amino acid residue.

In one embodiment, the 2A peptide is a T2A peptide. In one embodiment, the T2A peptide comprises the amino acid sequence SEQ ID NO: 11, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the sequence encoding the T2A peptide comprises the nucleotide sequence encoding the amino acid sequence SEQ ID NO: 11, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the sequence encoding the T2A peptide comprises the nucleotide sequence SEQ ID NO: 12, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

Alternatively, an Internal Ribosome Entry Site (IRES) may be used to link the chimeric MyD88 receptor and the sequence encoding at least an additional polypeptide sequence. IRES is an RNA element that allows for translation initiation in a cap-independent manner. IRES can link two coding sequences in one bicistronic vector and allow the translation of both proteins in cells. Non-limiting Examples of Chimeric MyD88 Receptors of the Present Disclosure

In one embodiment, the chimeric MyD88 receptor comprises the amino acid sequence of SEQ ID NO: 15, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the chimeric MyD88 receptor comprises the nucleotide sequence encoding the amino acid sequence of SEQ ID NO: 15, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the chimeric MyD88 receptor comprises the nucleotide sequence of SEQ ID NO: 16, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In one embodiment, the chimeric MyD88 receptor comprises the amino acid sequence of SEQ ID NO: 27, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the chimeric MyD88 receptor comprises the nucleotide sequence encoding the amino acid sequence of SEQ ID NO: 27, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the chimeric MyD88 receptor comprises the nucleotide sequence of SEQ ID NO: 28, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In one embodiment, the chimeric MyD88 receptor comprises the amino acid sequence of SEQ ID NO: 32, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the chimeric MyD88 receptor comprises the nucleotide sequence encoding the amino acid sequence of SEQ ID NO: 32, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the nucleotide sequence encoding the chimeric MyD88 receptor comprises the nucleotide sequence of SEQ ID NO: 33, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In one embodiment, the polynucleotide encodes the amino acid sequence of SEQ ID NO: 17, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the polynucleotide comprises the nucleotide sequence encoding the amino acid sequence of SEQ ID NO: 17, or an amino acid sequence having at least 50, at least 55, at least 60, at least 65, at least 70, at least 75, at least 80, at least 85, at least 90, at least 95, at least 96, at least 97, at least 98 or at least 99% sequence identity thereof. In one embodiment, the polynucleotide comprises the nucleotide sequence of SEQ ID NO: 18, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof.

In some embodiments, the polynucleotide encoding a chimeric MyD88 receptor is a DNA molecule. In some embodiments, the polynucleotide encoding a chimeric MyD88 receptor is an RNA molecule.

In another aspect, the present disclosure provides a chimeric MyD88 receptor encoded by the polynucleotide described herein.

### Vectors

In one aspect, the present disclosure provides recombinant vectors comprising a polynucleotide encoding a chimeric MyD88 receptor described above. In some embodiments, the recombinant vector comprises a polynucleotide encoding both a chimeric MyD88 receptor and an accessory gene (e.g., CAR) described above. In certain embodiments, the polynucleotide is operatively linked to at least one regulatory element for expression of the chimeric MyD88 receptor.

In certain embodiments, recombinant vectors of the invention comprise the nucleotide sequence of SEQ ID NO: 16, 18, 28, or 33, or a nucleotide sequence having at least 50, at least 55, at least 60, at least 65, at least 70, at least 75, at least 80, at least 85, at least 90, at least 95, at least 96, at least 97, at least 98 or at least 99% sequence identity thereof. In certain embodiments, recombinant vectors comprise a nucleotide sequence that encodes the amino acid sequence of SEQ ID NO: 15, 17, 27, or 32, or a variant having at least 50, at least 55, at least 60, at least 65, at least 70, at least 75, at least 80, at least 85, at least 90, at least 95, at least 96, at least 97, at least 98 or at least 99% sequence identity thereof.

In some embodiments, the vector is a viral vector. The viral vector may be a retroviral vector, an adenoviral vector, an adeno-associated virus vector, an alphaviral vector, a herpes virus vector, and a vaccinia virus vector. In some embodiments, the viral vector is a retroviral vector.

In some embodiments, the vector is a non-viral vector. The non-viral vector may be a plasmid (e.g., minicircle plasmid), a transposon (such as a PiggyBac- or a Sleeping Beauty transposon), or a single or double stranded DNA molecule that is used as a template for homology directed repair (HDR) based gene editing.

In certain embodiments, the polynucleotide encoding the chimeric MyD88 receptor is operably linked to at least a regulatory element. The regulatory element can be capable of mediating expression of the chimeric MyD88 receptor in the host cell. Regulatory elements include, but are not limited to, promoters, enhancers, initiation sites, polyadenylation (polyA) tails, IRES elements, response elements, and termination signals. In certain embodiments, the regulatory element regulates chimeric MyD88 receptor expression. In certain embodiments, the regulatory element increased the expression of the chimeric MyD88 receptor. In certain embodiments, the regulatory element increased the expression of the chimeric MyD88 receptor once the host cell is activated. In certain embodiments, the regulatory element decreases expression of the chimeric MyD88 receptor. In certain embodiments, the regulatory element decreases expression of the chimeric MyD88 receptor once the host cell is activated.

### Genetically Modified Host Cells

In one aspect, the present disclosure provides an isolated host cell comprising a chimeric MyD88 receptor described herein. In one aspect, the present disclosure provides an isolated host cell comprising a polynucleotide or a recombinant vector encoding a chimeric MyD88 receptor described herein.

In various embodiments, the host cell is an immune cell. In some embodiments, the host cell is a T cell, a natural killer (NK) cell, a mesenchymal stem cell (MSC), or a macrophage.

In some embodiments, the host cell is a T cell. T cells may include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells memory T cells, and NKT cells.

In some embodiments, the host cell is an up T-cell receptor (TCR) T-cell, a γδ T-cell, a CD8+ T-cell, a CD4+ T-cell, a cytotoxic T-cell, an invariant natural killer T (iNKT) cell, a memory T-cell, a memory stem T-cell (T_{SCM}), a naive T-cell, an effector T-cell, a T-helper cell, or a regulatory T-cell (Treg).

In various embodiments, the host cell is a NK cell. NK cell refers to a differentiated lymphocyte with a CD3− CD16+, CD3− CD56+, CD16+CD56+ and/or CD57+ TCR-phenotype.

In various embodiments, the host cell has been activated and/or expanded ex vivo.

In various embodiments, the host cell is an allogeneic cell with respect to the subject receiving the cell. In various embodiments, the host cell is an autologous cell with respect to the subject receiving the cell.

In some embodiments, the host cell is isolated from a subject having a cancer. In some embodiments, the host cell is derived from a blood, marrow, tissue, or a tumor sample.

In some embodiments, besides expressing a chimeric MyD88 receptor described herein, the host cell may further express a molecule that directs the host cell to a target cell. In some embodiments, the molecule that directs the host cell to a target cell is a chimeric antigen receptor (CAR), a T cell antigen coupler (TAC), an αβ TCR, or a bispecific antibody (e.g., bispecific T-cell engager (BiTE), or a dual-affinity re-targeting (DART) antibody).

In some embodiments, the molecule that redirects the host cell to target cells comprises an antigen-binding domain that specifically binds to an antigen. In some embodiments, the antigen is a tumor-associated antigen (TAA), an antigen expressed in the tumor stroma, an antigen expressed on endothelial cell, a virus-associated antigen or other pathogen-associated antigen, an antigen expressed on immune and/or stem cell, or an antigen associated with autoimmune disease (e.g., autoantigens or self-antigens).

Exemplary tumor-associated antigens (TAAs) that can be targeted by the modified host cells of the present disclosure include, but are not limited to, human epidermal growth factor receptor 2 (HER2), Eph receptor A2 (EphA2), B7-H3 (CD276), interleukin 13 receptor alpha 2 (IL13Rα2), cell surface GRP78, CD19, CD123, carbonic anhydrase, alpha-fetoprotein, A3, antigen specific for A33 antibody, Ba 733, BrE3-antigen, CA125, CD1, CDla, CD3, CD5, CD15, CD16, CD20, CD21, CD22, CD23, CD25, CD30, CD33, CD38, CD45, CD74, CD79a, CD80, CD138, collagen splice variants including COL11A1, colon-specific antigen-p (CSAp), CEA (CEACAM5), CEACAM6, Chlorotoxin, CSAp, EGFR, EGP-I, EGP-2, Ep-CAM, EphA1, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, insulin growth factor-1 (IGF-I), KC4-antigen, KS-1-antigen, KS1-4, Le-Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin C and its splice variants, TRAIL receptors, Tn antigen, Thomson-Friedenreich antigens, tumor necrosis antigens, VEGF, fibronectin and its splice variants including ED-B fibronectin, 17-1A-antigen, an angiogenesis marker, an oncogene marker or an oncogene product.

Additional tumor antigens that may be targeted by the modified host cells of the present disclosure include, but are not limited to, a kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLECl2A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRC5D), ganglioside GD3, ganglioside GM3, glycoceramide (GloboH), Glypican-3 (GPC3), Hepatitis A virus cellular receptor 1 (HAVCR1), hexasaccharide portion of globoH, high molecular weight-melanoma-associated antigen (HMWMAA), human Telomerase reverse transcriptase (hTERT), interleukin 11 receptor alpha (IL-11Ra), KIT (CD117), leukocyte-associated immunoglobulin-like receptor 1 (LAIR1), leukocyte immunoglobulin-like receptor subfamily A member 2 (LILRA2), Lewis(Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAX5), pannexin 3 (PANX3), placenta-specific 1 (PLACI), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEM1/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WT1), and X Antigen Family, Member 1A (XAGE1), or a fragment or variant thereof.

Exemplary antigens expressed in the tumor stroma that may be targeted by the modified host cells of the present disclosure include, but are not limited to oncofetal splice variants of fibronectin and tenascin C, tumor-specific splice variants of collagen, and fibroblast activating protein (FAP).

Exemplary antigens expressed on endothelial cells that may be targeted by the modified host cells of the present disclosure include, but are not limited to, VEGF receptors, and tumor endothelial markers (TEMs).

Exemplary virus-associated antigens that may be targeted by the modified host cells of the present disclosure include those derived from Adenoviridae (most adenoviruses); Arena viridae (hemorrhagic fever viruses); Birnaviridae; Bungaviridae (e.g., Hantaan viruses, bunga viruses, phleboviruses and Nairo viruses); Calciviridae (e.g., strains that cause gastroenteritis); Coronoviridae (e.g., coronaviruses); Filoviridae (e.g., ebola viruses); Flaviridae (e.g., dengue viruses, encephalitis viruses, yellow fever viruses); Hepadnaviridae (Hepatitis B virus; HBsAg); Herpesviridae (herpes simplex virus (HSV) 1 and 2, varicella zoster virus, cytomegalovirus (CMV), herpes virus); Iridoviridae (e.g., African swine fever virus); Norwalk and related viruses, and astroviruses; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvovirida (parvoviruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviurses and rotaviruses); Retroviridae (e.g., human immunodeficiency viruses, such as HIV-1 (also referred to as HTLV-III, LAV or HTLV-III/LAV, or HIV-III); and other isolates, such as HIV-LP); Rhabdoviradae (e.g., vesicular stomatitis viruses, rabies viruses); Togaviridae (e.g., equine encephalitis viruses, rubella viruses); and unclassified viruses (e.g., the etiological agents of Spongiform encephalopathies, the agent of delta hepatitis, the agents of non-A, non-B hepatitis (i.e. Hepatitis C)).

Additional infectious antigens that may be targeted by the modified host cells of the present disclosure include bacterial antigens, fungal antigens, parasite antigens, or prion antigens, or the like. Non-limiting examples of infectious bacteria include but are not limited to: Actinomyces israelli, Bacillus antracis, Bacteroides sp., Borelia burgdorferi, Chlamydia., Clostridium perfringers, Clostridium tetani, Corynebacterium diphtheriae, Corynebacterium sp., Enterobacter aerogenes, Enterococcus sp., Erysipelothrix rhusiopathiae, Fusobacterium nucleatum, Haemophilus influenzae, Helicobacter pyloris, Klebsiella pneumoniae, Legionella pneumophilia, Leptospira, Listeria monocytogenes, Mycobacteria sps. (e.g., M. tuberculosis, M. avium, M. gordonae, M. intracellulare, M. kansaii), Neisseria gonorrhoeae, Neisseria meningitidis, Pasteurella multocida, pathogenic Campylobacter sp., Rickettsia, Staphylococcus aureus, Streptobacillus monihformis, Streptococcus (anaerobic sps.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidium, and Treponema pertenue. Non-limiting examples of infectious fungi include: Cryptococcus neoformans, Histoplasma capsulatuin, Coccidioides immitis, Blastomyces dernatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma. Other medically relevant microorganisms have been descried extensively in the literature, e.g., see C. G. A. Thomas, “Medical Microbiology”, Bailliere Tindall, Great Britain 1983, which is hereby incorporated by reference in its entirety.

Other examples of antigens that may be targeted by the modified host cells of the present disclosure include antigens expressed on immune and/or stem cells to deplete these cells such as CD45RA and c-kit.

Further examples of antigens that may be targeted by the modified host cells of the present disclosure include antigens that are associated with an autoimmune disease, e.g., systemic lupus erythematosus, Wegener's granulomatosis, autoimmune hepatitis, Crohn's disease, scleroderma, ulcerative colitis, Sjögren's syndrome, Type 1 diabetes mellitus, uveitis, myocarditis, rheumatic fever, ankylosing spondylitis, rheumatoid arthritis, multiple sclerosis, and psoriasis.

In some embodiments, the host cell further expresses a molecule that directs the host cell to a target cell which is a chimeric antigen receptor (CAR). CARs are primarily comprised of 1) an antigen-binding moiety, such as a single-chain variable fragment (scFv) derived from an antigen-specific monoclonal antibody, and 2) a signaling domain, such as the ζ-chain from the T cell receptor CD3. These two regions are fused together via a transmembrane domain. A hinge domain is usually required to provide more flexibility and accessibility between the antigen-binding moiety and the transmembrane domain. Upon transduction, the lymphocyte expresses the CAR on its surface, and upon contact and ligation with the target antigen, it signals through the signaling domain (e.g., CD3 chain) inducing cytotoxicity and cellular activation. The antigen-binding moiety of the CAR may specifically bind to an antigen described above.

In addition, the CAR ectodomain might consist of a domain that can be paired with multiple, antigen recognition domains (e.g., avidin-CARs/biotin-labeled scFVs, CD16-CAR/MAbs, anti-FITC-CARs/FITC-labeled scFv, coiled-coil CARs (SUPRA CARs), anti-PNE-CARs/PNE-scFv, and NKG2D-CARs/ULBP2-MAb). These CARs are also known as ‘universal CARs’.

In some embodiments, the host cell has further been modified such that the expression and/or function of one or more gene(s) or gene product(s) in the host cell is reduced or eliminated. In some embodiments, the one or more gene(s) comprises IRAK3. In some embodiments, the one or more gene(s) comprises ZC3H12A (also known as Regnase-1 or MCPIP1). In some embodiments, the one or more gene(s) comprises IRAK3 and ZC3H12A.

In one aspect, provided herein is a method of generating the isolated host cell described herein. The method may comprise genetically modifying the host cell with a polynucleotide encoding the chimeric MyD88 receptor described herein or a recombinant vector comprising a polynucleotide encoding the chimeric MyD88 receptor described herein.

In some embodiments, the method of generating the isolated host cell described herein further comprises genetically modifying the host cell to expresses molecule that redirects the immune cell to target cells. In some embodiments, the molecule that directs the host cell to a target cell is a chimeric antigen receptor (CAR), a T cell antigen coupler (TAC), an UP TCR, or a bispecific antibody (e.g., bispecific T-cell engager (BiTE), or a dual-affinity re-targeting (DART) antibody). In some embodiments, the molecule that redirects the immune cell to target cells comprises an antigen-binding domain that specifically binds to an antigen described herein.

In some embodiments, the genetic modifying step is conducted via viral gene delivery. The viral vector may be a retroviral vector, a lentiviral vector, an adenoviral vector, an adeno-associated virus vector, an alphaviral vector, a herpes virus vector, a baculoviral vector, or a vaccinia virus vector.

In some embodiments, the genetic modifying step is conducted via non-viral gene delivery. The non-viral vector may be a plasmid (e.g., minicircle plasmid), a transposon (such as a PiggyBac- or a Sleeping Beauty transposon), or a single or double stranded DNA molecule that is used as a template for homology directed repair (HDR) based gene editing.

In some embodiments, the method of generating the isolated host cell described herein may further comprise modifying one or more gene(s) or gene product(s) in the host cell such that the expression and/or function of one or more gene(s) or gene product(s) in said host cell is reduced or eliminated. In some embodiments, the one or more gene(s) includes IRAK3. In some embodiments, the one or more gene(s) includes ZC3H12A (also known as Regnase-1 orMCPIPI). In some embodiments, the one or more gene(s) includes IRAK3 and ZC3H12A.

Methods for modifying one or more gene(s) or gene product(s) in the host cell may include disrupting the one or more gene(s) (e.g., IRAK3, ZC3H12A) with a site-specific nuclease. The term “site-specific nuclease” as used herein refers to a nuclease capable of specifically recognizing and cleaving a nucleic acid (DNA or RNA) sequence. Suitable site-specific nucleases for use in the present invention include, but are not limited to, an RNA-guided endonuclease (e.g., CRISPR-associated (Cas) proteins), a zinc finger nuclease, a TALEN nuclease, meganuclease, or a mega-TALEN nuclease.

Site-specific nucleases may create double-strand breaks (DSBs) or single-strand breaks (i.e., nicks) in a genomic DNA of a cell. Although not wishing to be bound by theory, these breaks are typically repaired by the cell using one of two mechanisms: non-homologous end joining (NHEJ) and homology-directed repair (HDR). In NHEJ, the double-strand breaks are repaired by direct ligation of the break ends to one another. As a result, no new nucleic acid material is inserted into the site, although a few bases may be lost or added, resulting in a small insertions and deletion (indel). In HDR, a donor polynucleotide with homology to the cleaved target DNA sequence is used as a template to repair the cleaved target DNA sequence, resulting in the transfer of genetic information from the donor polynucleotide to the target DNA. As such, new nucleic acid material may be inserted or copied into the cleavage site. In some cases, an exogenous donor polynucleotide can be provided to the cell. The modifications of the target DNA due to NHEJ and/or HDR may lead to, for example, gene correction, gene replacement, gene tagging, transgene insertion, nucleotide deletion, gene disruption, gene mutation, sequence replacement, etc. Accordingly, cleavage of DNA by a site-directed nuclease may be used to delete nucleic acid material from a target DNA sequence by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material (e.g., a transgene) into a locus of choice in the target DNA.

In some embodiments, the site-specific nuclease is an RNA-guided endonuclease. In particular, a group of RNA-guided endonucleases known as CRISPR-associated (Cas) proteins may be employed to genetically modify the T cell. A Cas protein may form an RNA-protein complex (referred to as RNP) with a guide RNA (gRNA) and is capable of cleaving a target site bearing sequence complementarity to a short sequence (typically about 20-40 nt) in the gRNA.

Examples of Cas proteins useful in the methods of the present disclosure include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In some embodiments, the Cas protein used in the methods described herein is a Cas9 protein. The Cas9 protein may be derived from S. pyogenes, Streptococcus thermophilus, Neisseria meningitidis, F. novicida, S. mutans or Treponema denticola.

Cas proteins useful in the methods of the present disclosure can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity.

A “guide RNA” or “gRNA” is an RNA molecule that binds to a Cas protein (e.g., Cas9 protein), or functional fragment or derivative thereof, and targets the Cas protein to a specific location within a target DNA. In some embodiments, the guide RNA is a single guide RNA (sgRNA). For Cas9, for example, a single-guide RNA can comprise a crRNA fused to a tracrRNA (e.g., via a linker). In some embodiments, the gRNA is designed to target a locus within or near the IRAK3 gene. In some embodiments, the gRNA is designed to target a locus within or near the ZC3H12A gene.

In some embodiments, modifying one or more gene(s) or gene product(s) in the host cell comprises silencing an mRNA transcribed from the one or more gene(s) (e.g., IRAK3, ZC3H12A) with an RNA interference (RNAi) molecule or an antisense oligonucleotide. In some embodiments, the RNAi molecule is a small interfering RNA (siRNA) or a small hairpin RNA (shRNA).

In some embodiments, modifying one or more gene(s) or gene product(s) in the host cell comprises inhibiting a protein expressed by the one or more gene(s) (e.g., IRAK3, ZC3H12A) with one or more of a small molecule inhibitor, a peptide, an antibody or antibody fragment, or an aptamer.

The genetically modifying step may be conducted ex vivo or in vivo. In some embodiments, the genetically modifying step is conducted ex vivo. The method may further include activation and/or expansion of the host cell ex vivo before, after and/or during the genetic modification.

In certain embodiments, host cells of the present invention may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., αβ TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, P2M, TAP1, TAP2, CIITA, RFX5, RFXAP and/or RFXANK locus.

Expression of the endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Isolation/Enrichment

The host cells may be autologous/autogeneic (“self”) or non-autologous (“non-self,” e.g., allogeneic, syngeneic, or xenogeneic). In certain embodiments, the host cells are obtained from a mammalian subject. In other embodiments, the host cells are obtained from a primate subject. In certain embodiments, the host cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, host cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In certain embodiments, the T lymphocytes for use in the compositions of the invention do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation/Activation

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Host cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T cell based host cells can be activated by binding to an agent that activates CD3ζ.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used.

In certain embodiments, the host cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 induce activation and/or expansion.

In general, the activating agents used in the present invention includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 M, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety.

In certain embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In certain embodiments, the NK based host cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Polynucleotide Transfer

In certain embodiments, the host cells are genetically modified to express a chimeric MyD88 receptor described above. The host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In order to genetically modify the host cell to express the chimeric MyD88 receptor, the polynucleotide construct encoding the chimeric MyD88 receptor must be transferred into the host cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue or organism.

In some embodiments, polynucleotides are transferred to the cell in a non-viral vector. In some embodiments, the non-viral vector is a transposon. Exemplary transposons hat can be used in the present invention include, but are not limited to, a sleeping beauty transposon and a PiggyBac transposon.

Nucleic acid vaccines can be used to transfer polynucleotides into the host cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the host cells can be genetically modified by methods ordinarily used by one of skill in the art. In certain embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993).

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity. In certain embodiments, the host cells may be removed from the subject and transfected ex vivo using the polynucleotides (e.g., expression vectors) of the invention. In certain embodiments, the host cells obtained from the subject can be transfected or transduced with the polynucleotides (e.g., expression vectors) of the invention and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprotatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. The term “site-specific nuclease” as used herein refers to a nuclease capable of specifically recognizing and cleaving a nucleic acid (DNA or RNA) sequence. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+ guide RNA) capable of inducing a double strand break in a target DNA sequence is introduced to a host cell, along with a donor polynucleotide encoding a chimeric MyD88 receptor of the present disclosure.

### Expansion/Proliferation

After the host cells are activated and transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120). Other illustrative examples for agents that may be used for the expansion of T cells are agents that bind to CD8, CD45 or CD90, such as αCD8, αCD45 or αCD90 antibodies. Illustrative examples of T cell population including antigen-specific T cells, T helper cells, cytotoxic T cells, memory T cell (an illustrative example of memory T-cells are CD62LICD81 specific central memory T cells) or regulatory T cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

After the host cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In certain embodiments, the binding agent includes antibodies (see for example Hoshino et al, Blood. 1991 Dec. 15; 78(12):3232-40.). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for host cell expansion include, but are not limited to, surfactant, piasmanate, pH buffers such as HEPES, and reducing agents such as N-acetyl-cysteine and 2-mercaptoethanol, antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

### Pharmaceutical Compositions

In some aspects, the present disclosure provides the compositions comprising the polypeptides of the chimeric MyD88 receptor, polynucleotides, vectors comprising same, and or cell compositions. Compositions of the present disclosure include pharmaceutical compositions.

In one aspect, the present disclosure provides a pharmaceutical composition comprising the genetically modified host cells described herein and a pharmaceutically acceptable carrier and/or excipient.

In another aspect, the present disclosure provides a pharmaceutical composition comprising a polynucleotide or a recombinant vector described herein, and a pharmaceutically accepted carrier and/or excipient.

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising genetically modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising genetically modified host cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the genetically modified host cells may be mixed with substances that adhere or penetrate then prior to their administration, e.g., but not limited to, nanoparticles.

### Therapeutic Methods

In one aspect, the present disclosure provides a method for killing a target cell. The method comprises contacting the target cell with the genetically modified host cell(s) or the pharmaceutical composition described herein. In some embodiments, the target cell is a cancer cell, a pathogen, or an auto-reactive immune cell.

In one aspect, the present disclosure provides a method for treating a disease in a subject in need thereof. In some embodiments, the method comprises administering to the subject a therapeutically effective amount of the genetically modified host cell(s) or the pharmaceutical composition described herein.

In some embodiments, the method comprises:
a) isolating T cells, NK cells, or macrophages from the subject;
b) genetically modifying said T cells, NK cells, or macrophages ex vivo with a polynucleotide encoding the chimeric MyD88 receptor described herein or a vector comprising the polynucleotide encoding the chimeric MyD88 receptor described herein;
c) optionally, genetically modifying said T cells, NK cells, or macrophages ex vivo to express a molecule that redirects said cells to a target cell and/or modifying the IRAK3 gene or gene product(s) thereof in said cells such that the expression and/or function of IRAK3 or gene product(s) thereof in said cells is reduced or eliminated;
c) optionally, expanding and/or activating said T cells, NK cells, or macrophages before, after or during step (b) or (c); and
d) introducing the genetically modified T cells, NK cells, or macrophages into the subject.

In some embodiments, the disease is cancer, an infectious disease, or an autoimmune disease.

In some embodiment, the disease is a cancer. The terms “cancer” and “cancerous” refer to or describe the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of cancer can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiment, the compositions and methods described in the present disclosure are used to treat an autoimmune disease. Non-limiting examples of autoimmune diseases that may be treated with the compositions and methods described herein include but are not limited to systemic lupus erythematosus, Wegener's granulomatosis, autoimmune hepatitis, Crohn's disease, scleroderma, ulcerative colitis, Sjögren's syndrome, Type 1 diabetes mellitus, uveitis, myocarditis, rheumatic fever, ankylosing spondylitis, rheumatoid arthritis, multiple sclerosis, and psoriasis.

In some embodiment, the compositions and methods described in the present disclosure are used to treat an infectious disease. Infectious diseases are well known to those skilled in the art, and non-limiting examples include but are not limited to infections of viral etiology such as HIV, influenza, Herpes, viral hepatitis, Epstein Bar, polio, viral encephalitis, measles, chicken pox, Papilloma virus; infections of bacterial etiology such as pneumonia, tuberculosis, syphilis; or infections of parasitic etiology such as malaria, trypanosomiasis, leishmaniasis, trichomoniasis, amoebiasis.

In some embodiments, the genetically modified host cell is an autologous cell. In some embodiments, the genetically modified host cell is an allogeneic cell. In cases where the host cell is isolated from a donor, the method may further include a method to prevent graft vs host disease (GVHD) and the immune cell rejection.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified immune cells. It is also contemplated that a variety of doses will be effective to improve in vivo effector function of modified immune cells.

In some embodiments, composition comprising the genetically modified host cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of genetically modified host immune cells will depend on the therapeutic use for which the composition is intended for.

Genetically modified host cells may be administered multiple times at dosages listed above. The genetically modified host cells may be allogeneic, syngeneic, xenogeneic, or autologous to the patient undergoing therapy.

The compositions and methods described in the present disclosure may be utilized in conjunction with other types of therapy for cancer, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the invention can be combined with other therapies that block inflammation (e.g., via blockage of IL1, INFα/β, IL6, TNF, IL23, etc.).

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating cancer, the compositions of the invention can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified immune cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

### EXAMPLES

The following examples are provided to further describe some of the embodiments disclosed herein. The examples are intended to illustrate, not to limit, the disclosed embodiments.

### Example 1. Rationale for Chimeric MyD88 Receptors

It was proposed that ideally, MyD88 costimulation should be directly linked to T cell activation or triggered by a molecule that normally inhibits T cell function. To explore both concepts, two prototype receptors were designed: i) an scfv-based MyD88 receptor that binds to IL-13 (α13-MyD88) as an example of a MyD88 receptor that is linked to T-cell activation (FIG. 1A), and ii) a PD1-MyD88 receptor that binds to PDL1, a molecule that is expressed on the cell surface of tumor cells and inhibits T cell function (FIG. 1B).

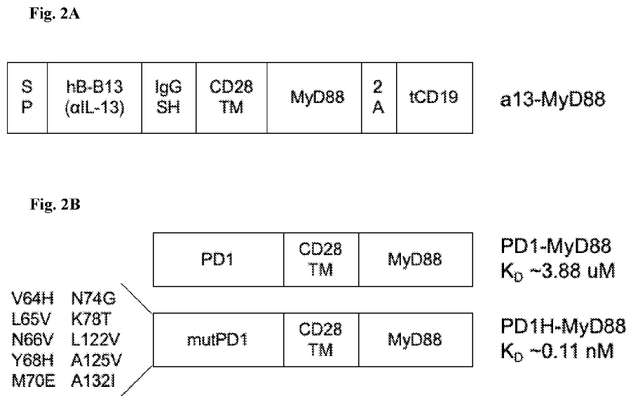

α13-MyD88: Activated CAR T cells produce large amounts of the Th2 cytokine IL-13, but most T cells do not express an IL-13 receptor (1, 2). In addition, several pro-tumorigenic roles for IL-13 have been described, including involvement in the epithelial-mesenchymal transition of tumor cells and polarization of macrophages to an M2 phenotype that enhances tumor invasiveness (3, 4). It was therefore concluded that it could be beneficial to make use of the cytokine with a chimeric receptor containing an IL-13-binding ectodomain and a MyD88 endodomain. An anti-IL-13-MyD88 receptor (ax13-MyD88) was developed with an scFv specific for human IL-13 (hB-B13), 12 amino-acid IgG1 short hinge, CD28 transmembrane (TM) domain, MyD88 endodomain, and tCD19 transduction marker (FIG. 2A).

PD1-MyD88: A second chimeric MyD88 receptor was developed to target the PD1/PDL1 axis. PD1 is a coinhibitory T cell receptors, made known by the success of “checkpoint blockade” and the FDA approval of the PD1 antibodies pembrolizumab and nivolumab for the treatment of several cancers including melanoma. When bound by its ligand PD-L1, PD1 inhibits PI3K activation and also recruits phosphatases that dephosphorylate TCR-proximal signaling components such as Zap70 and Lck, leading to inhibition of T cell proliferation, cytokine production, and cytotoxicity (7, 8) The approach used here sought to hijack this inhibitory signaling pathway and turn it into a costimulatory signal by creating a chimeric receptor with the PD1 ectodomain, a CD28 transmembrane domain, and a MyD88 endodomain (FIG. 2B). The affinity of wild-type PD1 for PDL1 is fairly low at 3.88 μM (12), which may be too low to provide optimal costimulatory signaling. Therefore two chimeric PD1-MyD88 receptors were generated, one (PD1-MyD88) with the wild-type PD1 ectodomain, and one (PD1H-MyD88) with mutations that increase the affinity of PD1 for PDL1 to 0.11 nM (FIG. 2B) (12).

### Example 2. Generation of Chimeric MyD88 Receptor T Cells

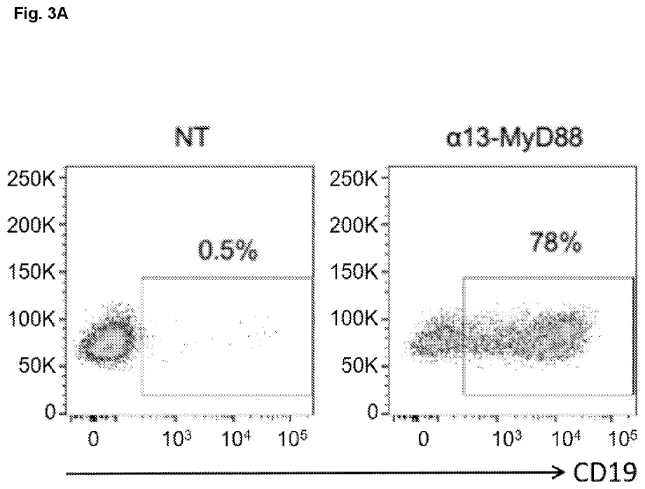

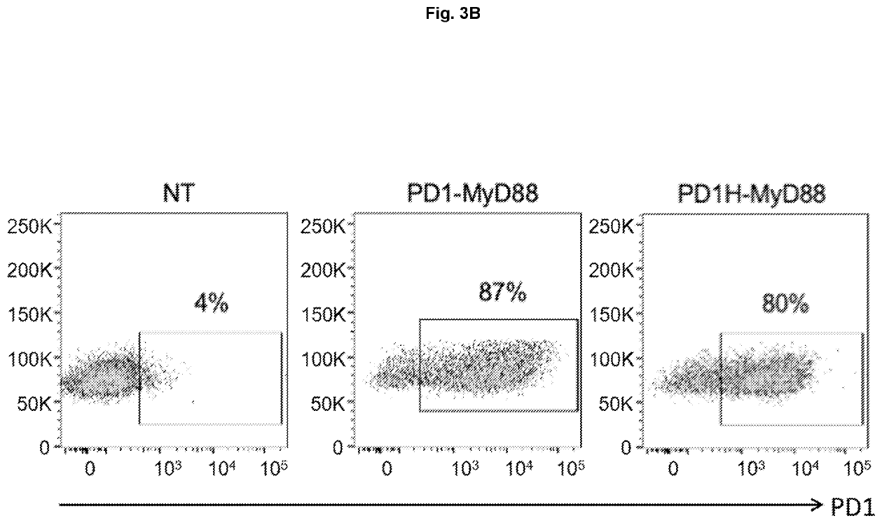

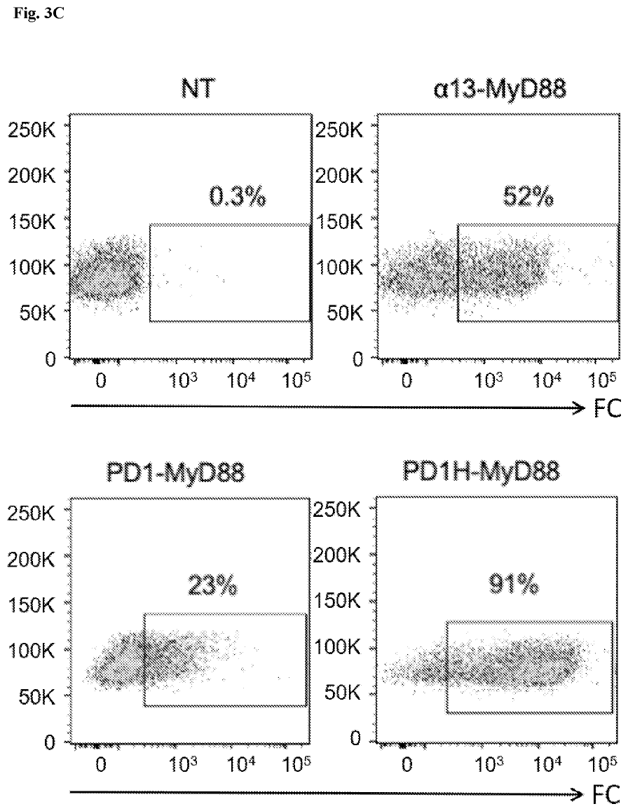

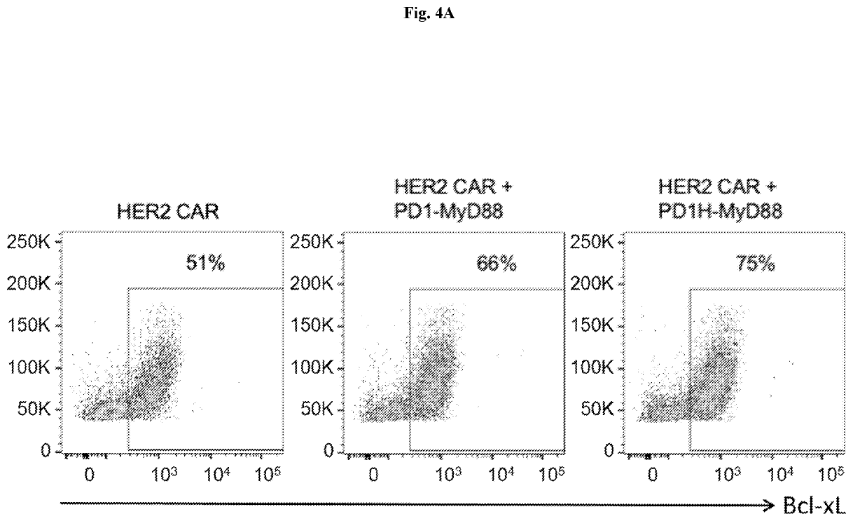

Chimeric MyD88 receptor T cells were generated by standard retroviral transduction. Flow cytometry was used to confirm that the chimeric MyD88 receptors is expressed on the T cell surface and that they bind their intended targets. After transduction, T cells were genetically modified as judged by detection of the tCD19 transduction marker for α13-MyD88 (FIG. 3A) or by direct detection for the PD1-MyD88 receptor (FIG. 3B). In order to confirm that the chimeric MyD88 receptors bind their intended targets, T cells were incubated with recombinant human IL-13-FC or PDL1-FC and then stained with an anti-FC antibody (FIG. 3C). Each of the receptors bound their intended target, but for the PD1-MyD88 receptors the high affinity PD1-MyD88 receptor was better able to bind the recombinant protein as judged by greater FC-positivity (both MFI and % positive cells) (FIG. 3C).

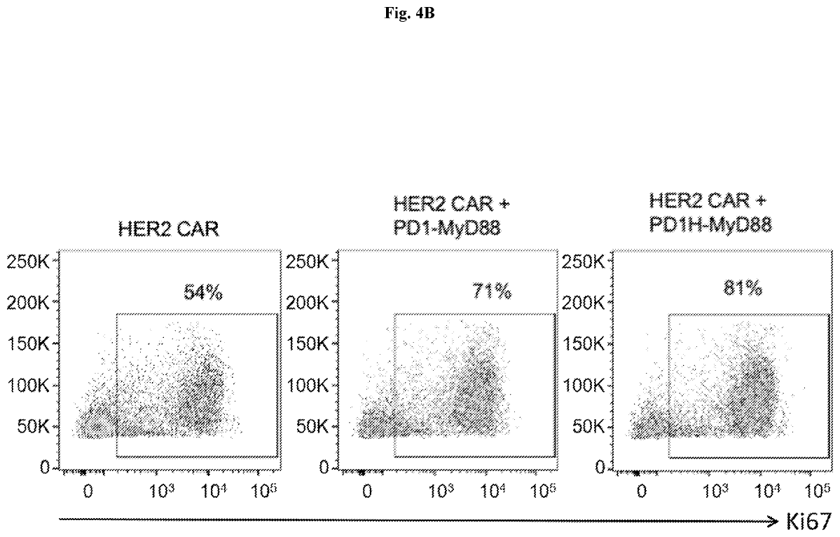

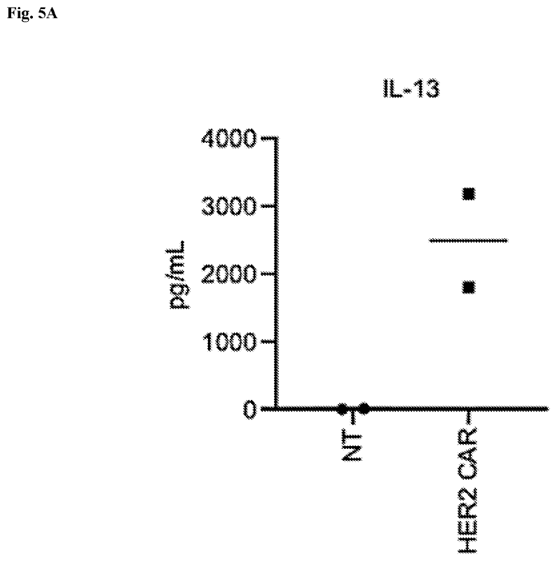

It was next investigated whether the PD1-MyD88 receptors would render T cells resistant to the inhibitory effects of exposure to PDL1. T cells were transduced with a HER2 CAR+/−PD1-MyD88 or PD1H-MyD88, stimulated with recombinant HER2 and PDL1 protein, and then stained for the anti-apoptotic protein Bcl-xL and the proliferative marker Ki67. These markers were chosen because PDL1 is known to repress Bcl-xL expression and inhibit proliferation (7). Co-expression of PD1-MyD88 or PD1H-MyD88 increased expression of both markers vs. CAR only, with the highest levels of Bcl-xL and Ki67 in T cells expressing HER2-CAR+PD1H-MyD88 (FIG. 4). These results suggest that the chimeric PD1-MyD88 receptors do indeed ameliorate the inhibitory signaling produced by the PD1/PDL1 axis. Based on these results PD1H-MyD88 was selected for future studies.

### Example 3. Chimeric MyD88 Receptors Improve CAR T Cell Function In Vitro and In Vivo

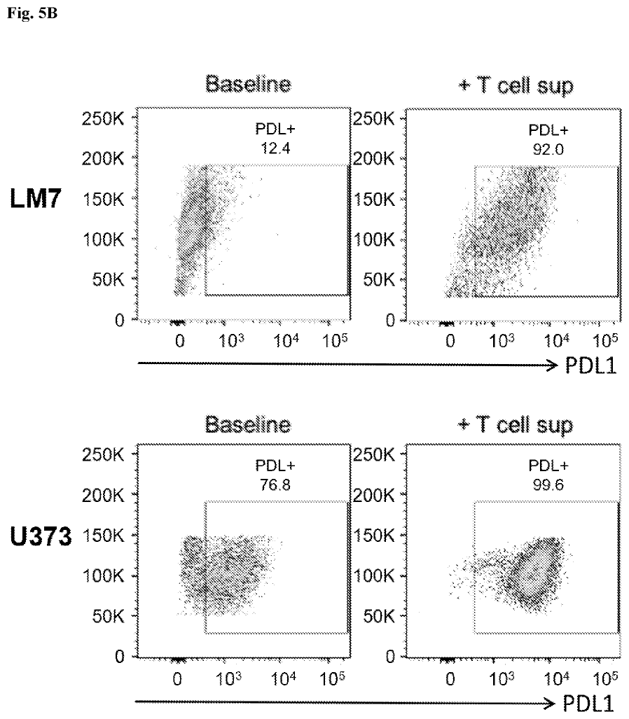

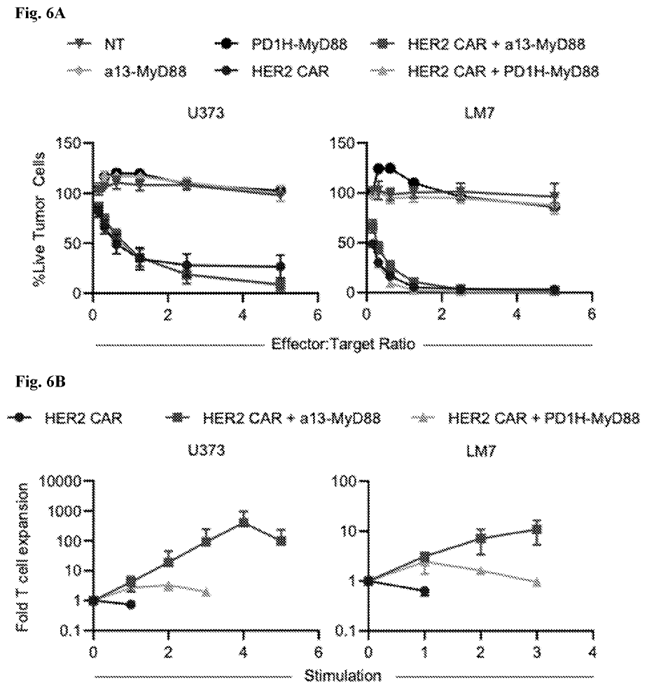

To get preliminary insight into the functionality of the chimeric MyD88 receptors, a model was used that consisted of HER2 CAR T cells as effector and LM7 and/or U373 cells as target cells. The suitability of the model was first confirmed by demonstrating that i) HER2.ζ CAR T cells produce IL-13 in a coculture assays (FIG. 5A) and ii) U373 and LM7 cells express PDL1 after exposure to supernatant from activated T cells (FIG. 5B). To ensure that the chimeric MyD88 receptors would not abrogate the cytolytic activity of HER2 CAR T cells, standard MTS assays were performed. When each receptor was co-expressed with a CAR, there was no discernible difference in 24-hour cytotoxicity vs. CAR alone (FIG. 6A). As expected, expression of α13-MyD88 or PD1H-MyD88 alone did not lead to tumor cell lysis (FIG. 6A).

Next the ability of the chimeric MyD88 receptors to sustain CAR T cell expansion was assessed using a standard restimulation assay. Expression of α13-MyD88 or PD1H-MyD88 receptors in HER2.CD28.ζ CAR T cells improved their ability to expand in comparison to parental HER2.CD28.ζ CAR T cells (FIG. 6B). In this initial experiment, α13-MyD88 receptor CAR T cells outperformed PD1H-MyD88 receptor CAR T cells. One potential explanation is that because CAR T cells produce IL-13 and tumor cells express PDL1, costimulatory signaling through the α13-MyD88 receptor is sustained after tumor cells have been killed whereas the signaling through PD1-MyD88 is not.

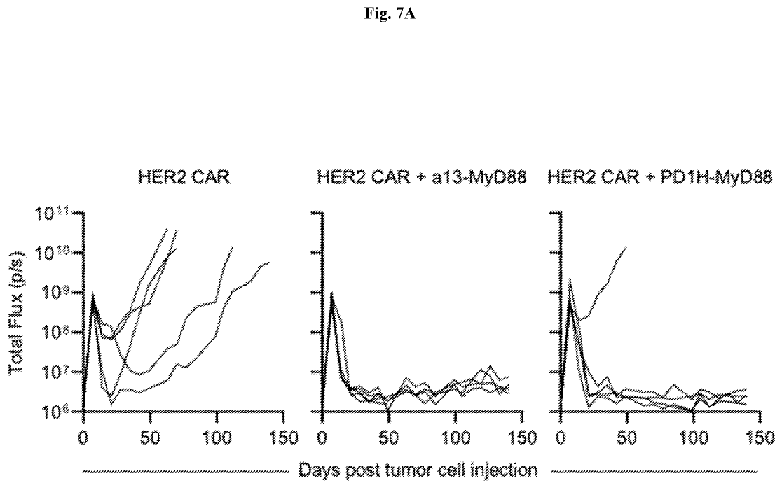

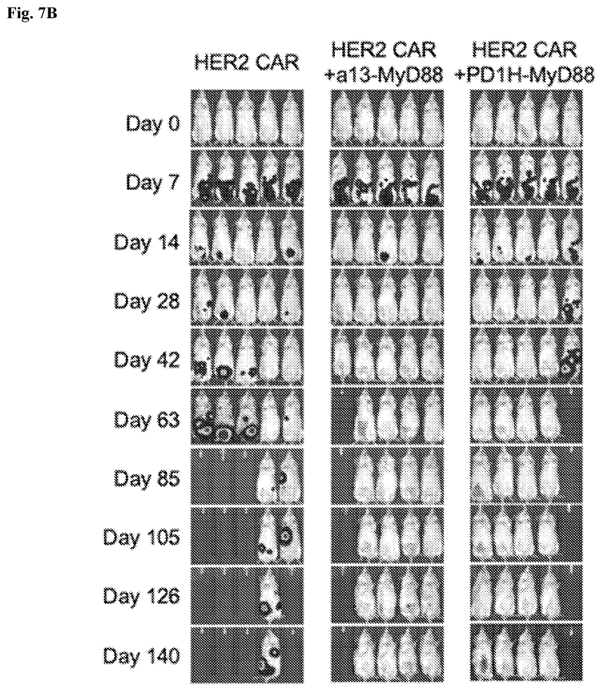

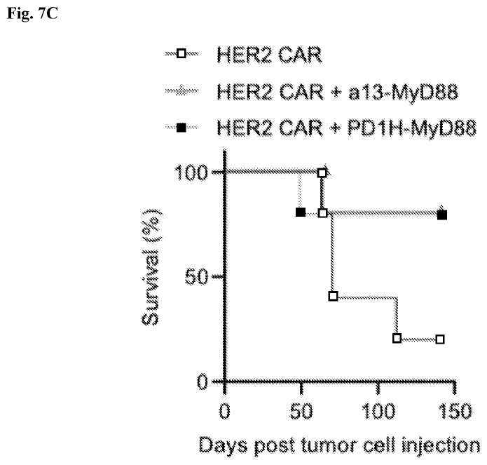

To demonstrate in vivo efficacy, NSG mice were injected intraperitoneally (i.p.) with 1×10⁶ LM7-ffluc tumor cells followed by low dose (1×10⁵) of HER2 CAR, α13-MyD88 HER2 CAR, or PD1H-MyD88 HER2 CAR T cells on day 7. Although HER2 CAR T cells had transient anti-tumor activity, 0/5 mice had complete responses. However, 4/5 mice receiving PD1H-MyD88 HER2 CAR and 5/5 mice receiving α13-MyD88 HER2 CAR completely cleared the tumor (FIGS. 7A, 7B). This led to a survival advantage of mice receiving chimeric MyD88 receptor CAR T cells (FIG. 7C).

### Example 4. Enhancing Chimeric MyD88 Receptors by Additional Genetic Modification

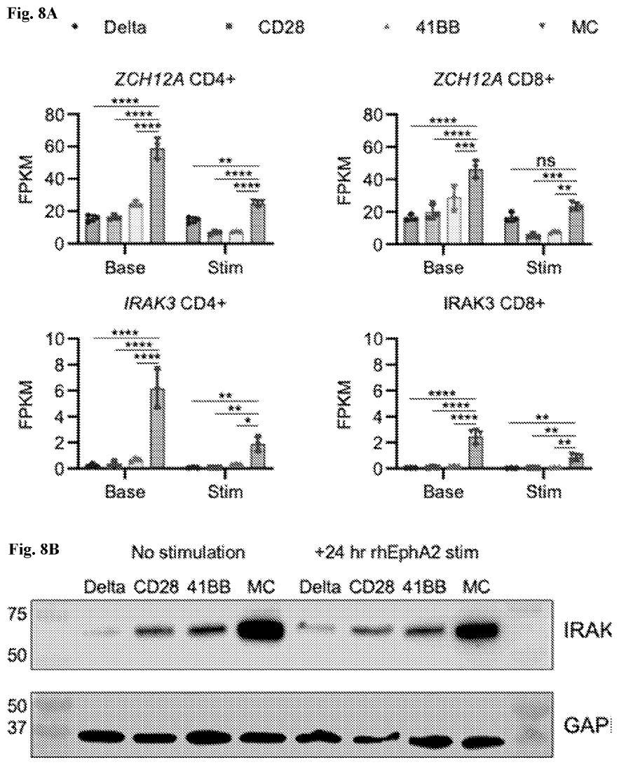

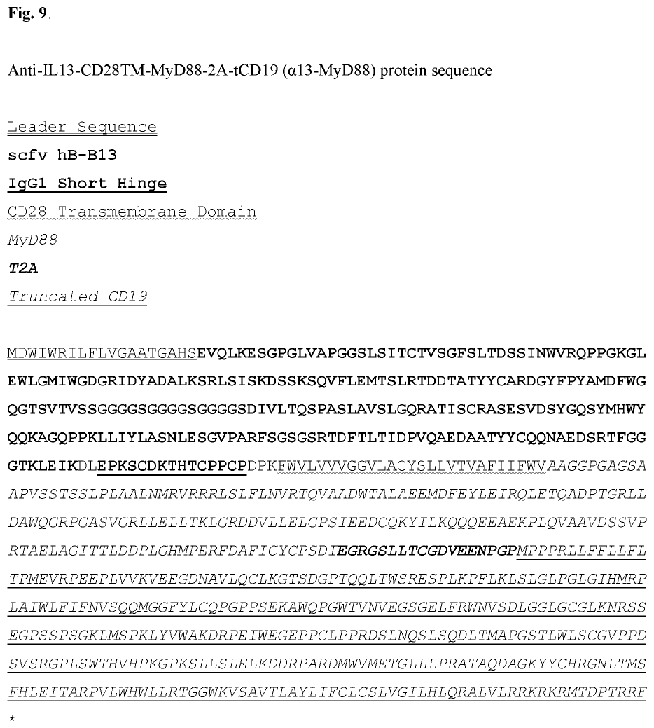

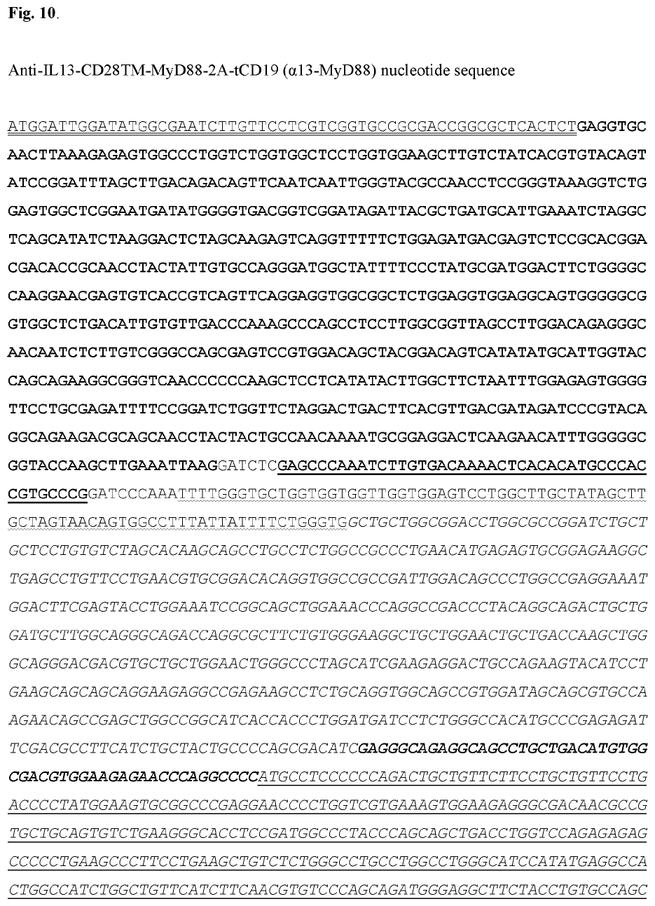

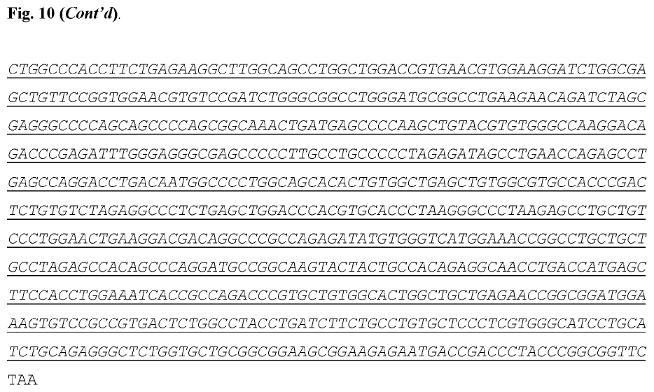

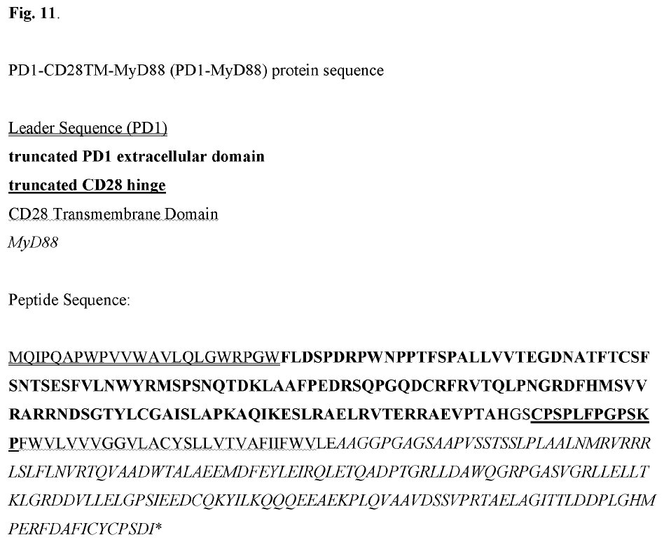

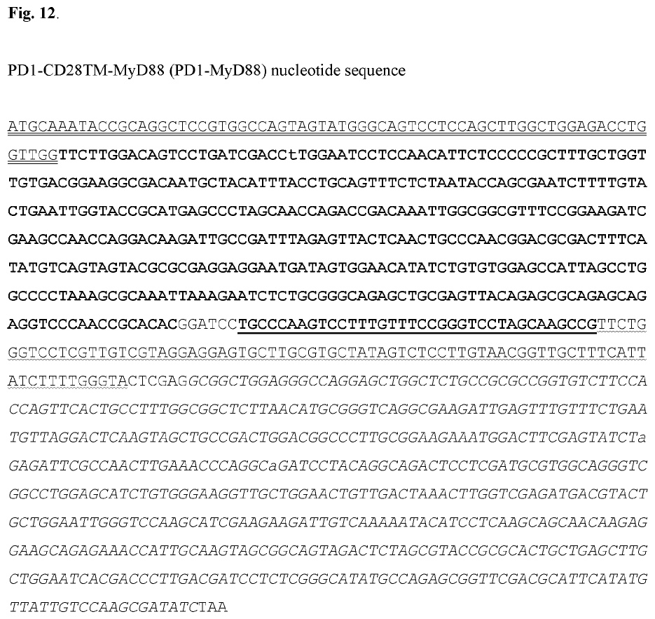

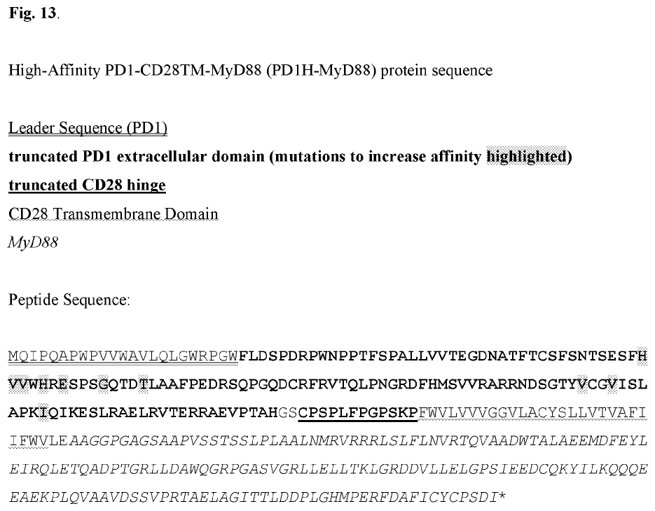

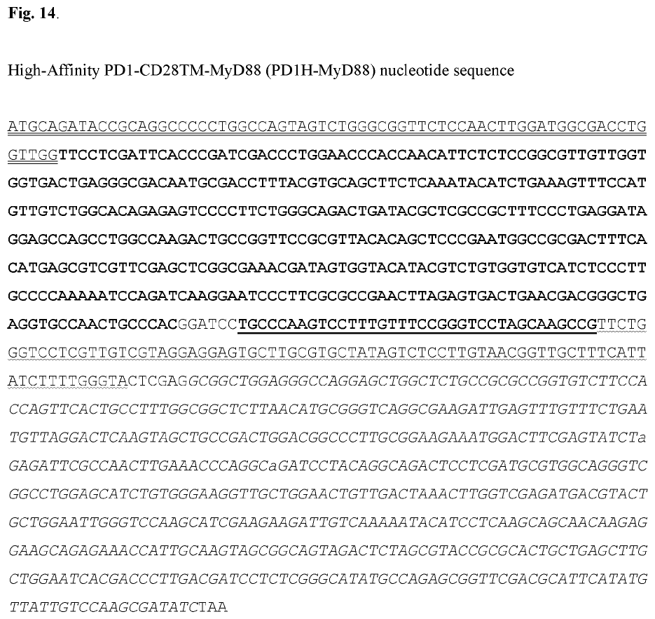

One avenue of increasing the potency of MyD88 signaling to knockout negative regulators of the MyD88 signaling pathway in T cells. T cells were previously generated that expressed EphA2-specific CARs with CD28.z (CD28), 4-1BB.z (4-1BB), MyD88.CD40.z (MC) signaling domains, and RNA seq analysis was performed post CAR activation. Differential expression of various genes was examined between MC− and CD28− or 4-1BB-CAR CD4+ and CD8+ T cells, wherein T cells were stimulated with LM7 tumor cells and RNAseq analysis was performed. Tables 1 to 4 show the top 25 differentially genes between MC− and CD28− or 4-1BB-CAR CD4+ and CD8+ T cells. Among the top differentially expressed genes were IRAK3 and ZC3H12A, both of which are known negative regulators of toll like receptor (TLR) and MyD88 signaling. FIG. 8A shows RNA levels for IRAK3 and ZC3H12A in unstimulated and stimulated MC-CAR T cells in comparison to Delta (non-functional)−, CD28−, or 4-1BB-CAR T cells. For IRAK3, this finding was also confirmed on the protein level with Western Blot (FIG. 8B). IRAK3 is a negative regulator of TLR signaling that functions by preventing the dissociation of IRAK1 and IRAK4 from MyD88, which in turn prevents downstream signaling, including activation of the NFκB pathway, from occurring (13). ZC3HI2A encodes MCPIP1, also known as Regnase-1, a ribonuclease induced by TLR and IL-1R signaling that targets several mRNA transcripts important for T cell activation such as c-Rel, OX40, ICOS, and IL-2 (14). Thus, the data demonstrates that knocking out IRAK3 and/or ZC3H12A in T cells that express chimeric MyD88 receptors of CARs with MyD88 signaling domains will further augment the benefit of MyD88 signaling.

TABLE 1
Top 25 differentially expressed genes upregulated in CD4+
MC-CAR T cells vs. 4-1BB-CAR T cells. T cells were stimulated
with LM7 tumor cells and RNAseq analysis was performed.

| | Genes High in CD4+ MC-CAR T cells | | | | | |
|---|---|---|---|---|---|---|
| | MC vs. 4-1BB | | | MC vs. CD28 | | |
| Gene | −logFC | p-value | FDR | −logFC | p-value | FDR |
| DENND5A | 2.75 | 1.93E−09 | 2.25E−05 | 0.36 | 4.85E−03 | 8.28E−03 |
| IRAK3 | 4.59 | 1.76E−08 | 4.98E−05 | 4.93 | 1.23E−08 | 7.60E−07 |
| MYO1B | 3.18 | 4.72E−08 | 5.71E−05 | 4.36 | 1.14E−08 | 7.54E−07 |
| RP11-215G15.5 | 1.97 | 6.18E−08 | 5.75E−05 | 3.67 | 1.37E−09 | 4.58E−07 |
| ULBP2 | 4.61 | 1.03E−07 | 5.76E−05 | 1.26 | 1.17E−04 | 3.32E−04 |
| ZC3H12A | 1.95 | 9.44E−08 | 5.76E−05 | 0.77 | 8.06E−05 | 2.45E−04 |
| SLAMF7 | 2.81 | 1.57E−07 | 5.84E−05 | 2.17 | 7.87E−07 | 7.18E−06 |
| PTPRK | 2.76 | 1.67E−07 | 5.93E−05 | 2.37 | 3.74E−07 | 4.41E−06 |
| FEZ1 | 4.87 | 2.21E−07 | 7.64E−05 | 6.67 | 1.17E−07 | 2.21E−06 |
| MGLL | 2.56 | 3.05E−07 | 9.39E−05 | 2.81 | 1.59E−07 | 2.63E−06 |
| USP6NL | 3.37 | 3.55E−07 | 1.03E−04 | 1.43 | 6.10E−05 | 1.93E−04 |
| SMPD3 | 4.53 | 5.22E−07 | 1.21E−04 | −4.63 | 1.05E−09 | 4.58E−07 |
| MGAT4A | 1.37 | 6.00E−07 | 1.35E−04 | −0.61 | 1.71E−04 | 4.56E−04 |
| ALCAM | 1.87 | 1.09E−06 | 2.12E−04 | 2.49 | 1.57E−07 | 2.62E−06 |
| KIAA1147 | 1.33 | 1.18E−06 | 2.20E−04 | −0.05 | 6.64E−01 | 7.01E−01 |
| CCDC141 | 2.56 | 1.21E−06 | 2.22E−04 | 0.66 | 5.27E−03 | 8.90E−03 |
| NLRC5 | 1.06 | 1.25E−06 | 2.28E−04 | −1.09 | 8.87E−07 | 7.76E−06 |
| PAQR8 | 1.62 | 1.50E−06 | 2.50E−04 | −1.29 | 2.29E−06 | 1.54E−05 |
| ACOXL | 2.42 | 1.57E−06 | 2.53E−04 | 4.74 | 4.45E−08 | 1.34E−06 |
| NFKBIZ | 3.17 | 1.60E−06 | 2.54E−04 | 0.79 | 7.15E−03 | 1.17E−02 |
| TNFRSF25 | 0.93 | 1.98E−06 | 2.91E−04 | 0.16 | 7.11E−02 | 9.35E−02 |
| UNC119 | 0.97 | 2.12E−06 | 3.06E−04 | 0.16 | 9.63E−02 | 1.23E−01 |
| ITGA1 | 2.32 | 2.38E−06 | 3.30E−04 | 1.04 | 3.57E−04 | 8.50E−04 |
| RHOBTB3 | 3.06 | 2.79E−06 | 3.76E−04 | 1.72 | 7.32E−05 | 2.25E−04 |
| IL17F | 4.81 | 4.22E−06 | 4.93E−04 | 8.40 | 4.28E−07 | 4.79E−06 |

TABLE 2
Top 25 differentially expressed genes downregulated in CD4+
MC-CAR T cells vs. 4-1BB-CAR T cells. T cells were stimulated
with LM7 tumor cells and RNAseq analysis was performed.

| | Genes Low in CD4+ MC-CAR T cells | | | | | |
|---|---|---|---|---|---|---|
| | MC vs. 4-1BB | | | MC vs. CD28 | | |
| Gene | −logFC | p-value | FDR | −logFC | p-value | FDR |
| LAG3 | −2.29 | 5.40E−08 | 5.71E−05 | 1.79 | 1.48E−06 | 1.10E−05 |
| P2RX7 | −2.61 | 9.63E−08 | 5.76E−05 | −3.27 | 1.30E−08 | 7.66E−07 |
| CTNS | −1.75 | 1.39E−07 | 5.78E−05 | 0.52 | 2.00E−03 | 3.78E−03 |
| PITPNM2 | −1.88 | 1.19E−07 | 5.78E−05 | −2.42 | 1.32E−08 | 7.73E−07 |
| RAMP1 | −2.56 | 1.25E−07 | 5.78E−05 | −1.62 | 5.61E−06 | 2.95E−05 |
| TJP2 | −1.78 | 1.40E−07 | 5.78E−05 | −0.29 | 3.83E−02 | 5.34E−02 |
| HAVCR2 | −2.09 | 1.61E−07 | 5.84E−05 | −0.33 | 3.74E−02 | 5.22E−02 |
| ADD2 | −2.55 | 4.13E−07 | 1.12E−04 | −0.51 | 3.10E−02 | 4.41E−02 |
| PCSK6 | −4.24 | 1.05E−06 | 2.09E−04 | −1.40 | 4.54E−03 | 7.80E−03 |
| IRF4 | −0.88 | 1.18E−06 | 2.20E−04 | 2.32 | 4.25E−10 | 4.44E−07 |
| IL10 | −5.27 | 1.41E−06 | 2.46E−04 | 0.77 | 2.00E−01 | 2.38E−01 |
| LGMN | −2.87 | 1.42E−06 | 2.46E−04 | −2.37 | 6.97E−06 | 3.50E−05 |
| MIR4435-1HG | −2.09 | 1.46E−06 | 2.49E−04 | −0.23 | 2.58E−01 | 3.00E−01 |
| CST7 | −1.97 | 1.73E−06 | 2.68E−04 | 0.06 | 7.35E−01 | 7.67E−01 |
| FAM3C | −1.05 | 2.60E−06 | 3.55E−04 | 0.69 | 1.14E−04 | 3.25E−04 |
| LRRC8C | −1.12 | 3.38E−06 | 4.31E−04 | −0.76 | 7.08E−05 | 2.19E−04 |
| EMP1 | −3.23 | 3.91E−06 | 4.79E−04 | −1.55 | 1.11E−03 | 2.26E−03 |
| ALS2CL | −3.59 | 4.04E−06 | 4.89E−04 | −7.76 | 6.32E−09 | 6.04E−07 |
| CEACAM21 | −1.41 | 4.47E−06 | 5.17E−04 | −2.65 | 1.94E−08 | 9.02E−07 |
| LATS2 | −1.60 | 4.50E−06 | 5.17E−04 | −1.96 | 7.57E−07 | 7.01E−06 |
| UBASH3B | −2.56 | 5.18E−06 | 5.56E−04 | −0.20 | 4.71E−01 | 5.15E−01 |
| SLA2 | −0.85 | 5.23E−06 | 5.57E−04 | −1.30 | 1.73E−07 | 2.76E−06 |
| TIMP1 | −1.23 | 5.42E−06 | 5.64E−04 | −2.35 | 2.46E−08 | 1.03E−06 |
| P2RX4 | −1.02 | 6.03E−06 | 5.93E−04 | −2.57 | 2.44E−09 | 4.67E−07 |
| GPR56 | −2.54 | 8.77E−06 | 7.53E−04 | 0.55 | 1.10E−01 | 1.39E−01 |

TABLE 3
Top 25 differentially expressed genes upregulated in CD8+
MC-CAR T cells vs. 4-1BB-CAR T cells. T cells were stimulated
with LM7 tumor cells and RNAseq analysis was performed.

| | Genes High in CD8+ MC-CAR T cells | | | | | |
|---|---|---|---|---|---|---|
| | MC vs. 4-1BB | | | MC vs. CD28 | | |
| Gene | −logFC | p-value | FDR | −logFC | p-value | FDR |
| CCR4 | 3.18 | 6.25E−09 | 8.66E−05 | 2.98063 | 1.03E−08 | 1.08E−05 |
| NFKBIZ | 2.40 | 4.73E−08 | 1.64E−04 | 3.55264 | 1.57E−09 | 5.43E−06 |
| ZC3H12A | 1.57 | 1.30E−07 | 2.99E−04 | 2.21491 | 4.23E−09 | 7.33E−06 |
| VNN2 | 1.55 | 6.47E−07 | 5.98E−04 | 1.16392 | 6.10E−06 | 5.43E−04 |
| ITGA1 | 2.38 | 6.97E−07 | 6.04E−04 | 1.06785 | 5.55E−04 | 9.70E−03 |
| USP6NL | 2.45 | 8.91E−07 | 6.50E−04 | 4.4413 | 1.11E−08 | 1.08E−05 |
| IRAK3 | 3.31 | 1.87E−06 | 8.94E−04 | 4.07881 | 2.79E−07 | 8.07E−05 |
| CD86 | 2.56 | 2.94E−06 | 1.04E−03 | 1.28476 | 7.11E−04 | 1.13E−02 |
| FOXP3 | 1.37 | 4.03E−06 | 1.30E−03 | 1.19113 | 1.32E−05 | 9.05E−04 |
| TNFSF4 | 2.25 | 5.52E−06 | 1.46E−03 | 1.76753 | 3.88E−05 | 1.76E−03 |
| ABTB2 | 2.29 | 5.35E−06 | 1.46E−03 | 1.23627 | 8.77E−04 | 1.29E−02 |
| NCF2 | 1.90 | 6.97E−06 | 1.54E−03 | 2.33204 | 1.14E−06 | 1.84E−04 |
| TNFSF15 | 2.54 | 7.01E−06 | 1.54E−03 | 1.94647 | 2.85E−05 | 1.40E−03 |
| STAT4 | 1.38 | 7.99E−06 | 1.61E−03 | 1.02013 | 1.12E−04 | 3.41E−03 |
| GPR55 | 1.72 | 8.66E−06 | 1.72E−03 | 1.92495 | 2.51E−06 | 2.98E−04 |
| CCNI2 | 1.60 | 2.23E−05 | 3.33E−03 | 2.4349 | 4.67E−07 | 1.10E−04 |
| P2RX5 | 1.88 | 3.96E−05 | 4.54E−03 | 2.38655 | 4.83E−06 | 4.61E−04 |
| GCNT2 | 1.86 | 4.21E−05 | 4.74E−03 | 1.87812 | 2.80E−05 | 1.38E−03 |
| RXRA | 1.23 | 5.52E−05 | 5.69E−03 | 1.36354 | 1.65E−05 | 1.02E−03 |
| IGFBP5 | 4.04 | 5.94E−05 | 5.87E−03 | 4.04984 | 5.77E−05 | 2.26E−03 |
| IFI44L | 3.44 | 6.14E−05 | 5.99E−03 | 8.15229 | 1.05E−05 | 8.03E−04 |
| SAMD4A | 1.97 | 8.88E−05 | 7.46E−03 | 2.75922 | 5.96E−06 | 5.40E−04 |
| ACVRIC | 2.11 | 9.00E−05 | 7.49E−03 | 1.8992 | 1.71E−04 | 4.56E−03 |
| MOB3B | 1.73 | 1.16E−04 | 8.38E−03 | 1.65419 | 1.37E−04 | 3.93E−03 |
| RP11-445H22.3 | 1.17 | 1.51E−04 | 9.87E−03 | 2.76832 | 1.98E−07 | 7.17E−05 |

TABLE 4
Top 25 differentially expressed genes downregulated in CD8+
MC-CAR T cells vs. 4-1BB-CAR T cells. T cells were stimulated
with LM7 tumor cells and RNAseq analysis was performed.

| | Genes Low in CD8+ MC-CAR T cells | | | | | |
|---|---|---|---|---|---|---|
| | MC vs. 4-1BB | | | MC vs. CD28 | | |
| Gene | −logFC | p-value | FDR | −logFC | p-value | FDR |
| PITPNM2 | −1.69 | 4.45E−08 | 1.64E−04 | −1.89 | 1.22E−08 | 1.08E−05 |
| PTMS | −1.63 | 4.22E−08 | 1.64E−04 | −1.24 | 7.41E−07 | 1.43E−04 |
| LAG3 | −1.67 | 3.81E−07 | 5.20E−04 | −1.46 | 1.44E−06 | 2.07E−04 |
| CCL4L2 | −3.38 | 3.58E−07 | 5.20E−04 | −2.85 | 2.03E−06 | 2.67E−04 |
| KIAA1324 | −2.20 | 4.50E−07 | 5.20E−04 | −1.75 | 4.76E−06 | 4.59E−04 |
| GZMB | −2.09 | 5.82E−07 | 5.96E−04 | −1.42 | 2.45E−05 | 1.26E−03 |
| ADTRP | −2.05 | 8.23E−07 | 6.50E−04 | −1.01 | 5.85E−04 | 9.97E−03 |
| LBH | −1.55 | 1.20E−06 | 7.95E−04 | −1.27 | 8.83E−06 | 7.12E−04 |
| UBASH3B | −1.14 | 1.78E−06 | 8.86E−04 | −1.77 | 1.78E−08 | 1.23E−05 |
| SMOX | −1.25 | 1.64E−06 | 8.86E−04 | −1.85 | 2.62E−08 | 1.58E−05 |
| EMP1 | −3.35 | 2.43E−06 | 9.91E−04 | −3.39 | 2.13E−06 | 2.76E−04 |
| ADD2 | −2.27 | 2.39E−06 | 9.91E−04 | −1.97 | 9.53E−06 | 7.46E−04 |
| CCL4 | −3.14 | 2.33E−06 | 9.91E−04 | −2.47 | 2.33E−05 | 1.21E−03 |
| CCL4L1 | −3.39 | 3.33E−06 | 1.13E−03 | −2.83 | 1.92E−05 | 1.09E−03 |
| TJP2 | −1.47 | 4.77E−06 | 1.44E−03 | −1.65 | 1.40E−06 | 2.06E−04 |
| RUSC2 | −2.10 | 6.22E−06 | 1.46E−03 | −1.88 | 1.82E−05 | 1.05E−03 |
| PTCH1 | −2.26 | 6.74E−06 | 1.54E−03 | −2.00 | 2.09E−05 | 1.13E−03 |
| HAVCR2 | −1.02 | 7.78E−06 | 1.61E−03 | −1.40 | 3.39E−07 | 9.07E−05 |
| GPR56 | −2.22 | 1.11E−05 | 2.11E−03 | −1.50 | 3.77E−04 | 7.52E−03 |
| ITGAX | −1.48 | 1.37E−05 | 2.40E−03 | −1.50 | 1.11E−05 | 8.12E−04 |
| IFITM10 | −3.73 | 1.42E−05 | 2.40E−03 | −3.37 | 3.68E−05 | 1.69E−03 |
| MB | −3.58 | 1.39E−05 | 2.40E−03 | −2.70 | 1.83E−04 | 4.72E−03 |
| C4orf48 | −1.31 | 1.68E−05 | 2.73E−03 | −1.06 | 1.08E−04 | 3.31E−03 |
| GBP2 | −1.24 | 1.71E−05 | 2.76E−03 | −1.01 | 1.06E−04 | 3.28E−03 |
| MAP3K6 | −1.56 | 2.05E−05 | 3.16E−03 | −1.20 | 2.23E−04 | 5.30E−03 |

### REFERENCES

1. de Vries J E. The role of IL-13 and its receptor in allergy and inflammatory responses. J Allergy Clin Immunol. 1998; 102(2):165-9.
2. Newcomb D C, Boswell M G, Zhou W, Huckabee M M, Goleniewska K, Sevin C M, et al. Human TH17 cells express a functional IL-13 receptor and IL-13 attenuates IL-17A production. J Allergy Clin Immunol. 2011; 127(4):1006-13 el-4.
3. Little A C, Pathanjeli P, Wu Z, Bao L, Goo L E, Yates J A, et al. IL-4/IL-13 Stimulated Macrophages Enhance Breast Cancer Invasion Via Rho-GTPase Regulation of Synergistic VEGF/CCL-18 Signaling. Front Oncol. 2019; 9:456.
4. Cao H, Zhang J, Liu H, Wan L, Zhang H, Huang Q, et al. IL-13/STAT6 signaling plays a critical role in the epithelial-mesenchymal transition of colorectal cancer cells. Oncotarget. 2016; 7(38):61183-98.
5. Chang Z L, Lorenzini M H, Chen X, Tran U, Bangayan N J, Chen Y Y. Rewiring T-cell responses to soluble factors with chimeric antigen receptors. Nat Chem Biol. 2018; 14(3):317-24.
6. Hou A J, Chang Z L, Lorenzini M H, Zah E, Chen Y Y. TGF-beta-responsive CAR-T cells promote anti-tumor immune function. Bioeng Transl Med. 2018; 3(2):75-86.
7. Riley J L. PD-1 signaling in primary T cells. Immunol Rev. 2009; 229(1):114-25.
8. Parry R V, Chemnitz J M, Frauwirth K A, Lanfranco A R, Braunstein I, Kobayashi S V, et al. CTLA-4 and PD-1 receptors inhibit T-cell activation by distinct mechanisms. Molecular and cellular biology. 2005; 25(21):9543-53.
9. Ankri C, Shamalov K, Horovitz-Fried M, Mauer S, Cohen C J. Human T cells engineered to express a programmed death 1/28 costimulatory retargeting molecule display enhanced antitumor activity. J Immunol. 2013; 191(8):4121-9.
10. Liu X, Ranganathan R, Jiang S, Fang C, Sun J, Kim S, et al. A Chimeric Switch-Receptor Targeting PD1 Augments the Efficacy of Second-Generation CAR T Cells in Advanced Solid Tumors. Cancer research. 2016; 76(6):1578-90.
11. Sukumaran S, Watanabe N, Bajgain P, Raja K, Mohammed S, Fisher W E, et al. Enhancing the Potency and Specificity of Engineered T Cells for Cancer Treatment. Cancer discovery. 2018; 8(8):972-87.
12. Maute R L, Gordon S R, Mayer A T, McCracken M N, Natarajan A, Ring N G, et al. Engineering high-affinity PD-1 variants for optimized immunotherapy and immuno-PET imaging. Proceedings of the National Academy of Sciences of the United States of America. 2015;112(47):E6506-14.
13. Kobayashi K, Hernandez L D, Galan J E, Janeway C A, Jr., Medzhitov R, Flavell R A. IRAK-M is a negative regulator of Toll-like receptor signaling. Cell. 2002; 110(2):191-202.
14. Mao R, Yang R, Chen X, Harhaj E W, Wang X, Fan Y. Regnase-1, a rapid response ribonuclease regulating inflammation and stress responses. Cell Mol Immunol. 2017; 14(5):412-22.
15. United States Patent Application No. 20130243777A1

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A polynucleotide encoding a chimeric MyD88 receptor comprising:
a) an extracellular domain comprising a target-binding moiety that binds to a target molecule;
b) a transmembrane domain; and
c) a cytoplasmic domain comprising a MyD88 polypeptide or a functional fragment thereof.

2. The polynucleotide of claim 1, wherein the MyD88 polypeptide comprises the amino acid sequence of SEQ ID NO: 9 or 44, or an amino acid sequence having at least 80% identity thereof; or the nucleotide sequence encoding the MyD88 polypeptide comprises the nucleotide sequence of SEQ ID NO: 10, 26 or 45, or a nucleotide sequence having at least 80% identity thereof.

3-5. (canceled)

6. The polynucleotide of claim 1, wherein the target molecule is a molecule secreted by an immune cell expressing the chimeric MyD88 receptor.

7. The polynucleotide of claim 1, wherein the target molecule is interleukin 5 (IL-5), IL-6, IL-10, IL-13, IL-17, GM-CSF, RANTES (CCL5), OX40, ICOS, programmed death-ligand 1 (PD-L1), Galectin-9, MHC class II, CD48, CD155, or CD112.

8. The polynucleotide of claim 1, wherein the target molecule is IL-13 or programmed death-ligand 1 (PD-L1).

9. The polynucleotide of claim 1, wherein the target molecule is expressed by a cell not expressing the chimeric MyD88 receptor.

10. The polynucleotide of claim 9, wherein the cell is an immune cell, cancer cell, and/or stromal cell.

11-12. (canceled)

13. The polynucleotide of claim 1, wherein the target-binding moiety is an antibody or an antibody fragment or is derived from a cell surface receptor.

14. The polynucleotide of claim 1, wherein the target-binding moiety is a single chain variable fragment (scFv).

15. (canceled)

16. The polynucleotide of claim 1, wherein the target-binding moiety comprises an ectodomain of a cell surface receptor, or a functional variant or fragment thereof.

17. The polynucleotide of claim 1, wherein the target-binding moiety is an anti-IL-13 scFv.

18. (canceled)

19. The polynucleotide of claim 17, wherein the anti-IL-13 scFv comprises the amino acid sequence SEQ ID NO: 3, or an amino acid sequence having at least 80% identity thereof; or the nucleotide sequence encoding the anti-IL-13 scFv comprises the nucleotide sequence SEQ ID NO: 4, or a nucleotide sequence having at least 80% identity thereof.

20. (canceled)

21. The polynucleotide of claim 1, wherein the target-binding moiety is derived from PD1, TIM3, LAG3, 2B4, or TIGIT.

22. The polynucleotide of claim 21, wherein the target-binding moiety comprises an ectodomain of PD1, or a functional variant or fragment thereof.

23. The polynucleotide of claim 22, wherein the target-binding moiety derived from PD1 comprises the amino acid sequence of SEQ ID NO: 21, or SEQ ID NO: 30, or an amino acid sequence having at least 80% identity thereof; or the nucleotide sequence encoding the target-binding moiety derived from PD1 comprises the nucleotide sequence of SEQ ID NO: 22, or SEQ ID NO: 31, or a nucleotide sequence having at least 80% identity thereof.

24. (canceled)

25. The polynucleotide of claim 1, wherein the transmembrane domain is derived from CD28, CD8, CD4, CD3ζ, CD40, CD134 (OX-40), CD19, or CD7.

26-28. (canceled)

29. The polynucleotide of claim 1, wherein the extracellular domain further comprises a hinge domain between the target-binding moiety and the transmembrane domain.

30. The polynucleotide of claim 29, wherein the hinge domain is derived from IgG1, IgG4, CD28, or CD8.

31-36. (canceled)

37. The polynucleotide of claim 1, wherein the extracellular domain further comprises a leader sequence.

38. The polynucleotide of claim 37, wherein the leader sequence is derived from CD8α, PD1, or human immunoglobulin heavy chain variable region.

39-42. (canceled)

43. The polynucleotide of claim 1, wherein the chimeric MyD88 receptor comprises the amino acid sequence of SEQ ID NO: 15, 27, or 32, or an amino acid sequence having at least 80% sequence identity thereof; or the nucleotide sequence encoding the chimeric MyD88 receptor comprises the nucleotide sequence of SEQ ID NO: 16, 28 or 33, or a nucleotide sequence having at least 80% sequence identity thereof.

44-48. (canceled)

49. The polynucleotide of claim 1, wherein the polynucleotide further encodes at least one additional polypeptide.

50. The polynucleotide of claim 49, wherein the at least one polypeptide is a transduced host cell selection marker, an in vivo tracking marker, a cytokine, or a safety switch gene.

51-53. (canceled)

54. The polynucleotide of claim 49, wherein the sequence encoding the chimeric MyD88 receptor is operably linked to the sequence encoding at least an additional polypeptide sequence via a sequence encoding a self-cleaving peptide and/or an internal ribosomal entry site (IRES).

55-63. (canceled)

64. A chimeric MyD88 receptor encoded by the polynucleotide of claim 1.

65. A recombinant vector comprising the polynucleotide of claim 1.

66-70. (canceled)

71. An isolated host cell comprising the polynucleotide of claim 1 or a recombinant vector comprising the polynucleotide.

72-90. (canceled)

91. A pharmaceutical composition comprising the host cell of claim 71 and a pharmaceutically acceptable carrier and/or excipient.

92. A method of generating the isolated host cell of claim 71, said method comprising genetically modifying the host cell with the polynucleotide or a recombinant vector comprising the polynucleotide.

93-100. (canceled)

101. A method for killing a target cell, said method comprising contacting said cell with the host cell(s) of claim 71 or the pharmaceutical composition comprising the host cell and a pharmaceutically acceptable carrier and/or excipient.

102. A method for treating a disease in a subject in need thereof, said method comprising administering to the subject a therapeutically effective amount of the host cell(s) of claim 71 or the pharmaceutical composition comprising the host cell and a pharmaceutically acceptable carrier and/or excipient.

103-106. (canceled)


SEQUENCE LISTING (45 sequences)

SEQ ID NO: 1 - protein, 19 aa, Artificial Sequence
  Description of Artificial Sequence Synthetic peptide
MDWIWRILFLVGAATGAHS

SEQ ID NO: 2 - DNA, 57 bp, Artificial Sequence
  Description of Artificial Sequence Synthetic oligonucleotide
atggattggatatggcgaatcttgttcctcgtcggtgccgcgaccggcgctcactct

SEQ ID NO: 3 - protein, 246 aa, Artificial Sequence
  Description of Artificial Sequence Synthetic polypeptide
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSGGGGSDIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKDL

SEQ ID NO: 4 - DNA, 738 bp, Artificial Sequence
  Description of Artificial Sequence Synthetic polynucleotide
gaggtgcaacttaaagagagtggccctggtctggtggctcctggtggaagcttgtctatcacgtgtacagtatccggatttagcttgacagacagttcaatcaattgggtacgccaacctccgggtaaaggtctggagtggctcggaatgatatggggtgacggtcggatagattacgctgatgcattgaaatctaggctcagcatatctaaggactctagcaagagtcaggtttttctggagatgacgagtctccgcacggacgacaccgcaacctactattgtgccagggatggctattttccctatgcgatggacttctggggccaaggaacgagtgtcaccgtcagttcaggaggtggcggctctggaggtggaggcagtgggggcggtggctctgacattgtgttgacccaaagcccagcctccttggcggttagccttggacagagggcaacaatctcttgtcgggccagcgagtccgtggacagctacggacagtcatatatgcattggtaccagcagaaggcgggtcaaccccccaagctcctcatatacttggcttctaatttggagagtggggttcctgcgagattttccggatctggttctaggactgacttcacgttgacgatagatcccgtacaggcagaagacgcagcaacctactactgccaacaaaatgcggaggactcaagaacatttgggggcggtaccaagcttgaaattaaggatctc

SEQ ID NO: 5 - protein, 15 aa, Artificial Sequence
  Description of Artificial Sequence Synthetic peptide
EPKSCDKTHTCPPCP

SEQ ID NO: 6 - DNA, 45 bp, Artificial Sequence
  Description of Artificial Sequence Synthetic oligonucleotide
gagcccaaatcttgtgacaaaactcacacatgcccaccgtgcccg

SEQ ID NO: 7 - protein, 27 aa, Artificial Sequence
  Description of Artificial Sequence Synthetic peptide
FWVLVVVGGVLACYSLLVTVAFIIFWV

SEQ ID NO: 8 - DNA, 81 bp, Artificial Sequence
  Description of Artificial Sequence Synthetic oligonucleotide
ttttgggtgctggtggtggttggtggagtcctggcttgctatagcttgctagtaacagtggcctttattattttctgggtg

SEQ ID NO: 9 - protein, 171 aa, Artificial Sequence
  Description of Artificial Sequence Synthetic polypeptide
AAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 10 - DNA, 513 bp, Artificial Sequence
  Description of Artificial Sequence Synthetic polynucleotide
gctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatc

SEQ ID NO: 11 - protein, 18 aa, Thosea asigna virus
EGRGSLLTCGDVEENPGP

SEQ ID NO: 12 - DNA, 54 bp, Thosea asigna virus
gagggcagaggcagcctgctgacatgtggcgacgtggaagagaacccaggcccc

SEQ ID NO: 13 - protein, 333 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 14 - DNA, 999 bp, Artificial Sequence
  Description of Artificial Sequence Synthetic polynucleotide
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 15 - protein, 481 aa, Artificial Sequence
  Description of Artificial Sequence Synthetic polypeptide
MDWIWRILFLVGAATGAHSEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSGGGGSDIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKDLEPKSCDKTHTCPPCPDPKFWVLVVVGGVLACYSLLVTVAFIIFWVAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 16 - DNA, 1443 bp, Artificial Sequence
  Description of Artificial Sequence Synthetic polynucleotide
atggattggatatggcgaatcttgttcctcgtcggtgccgcgaccggcgctcactctgaggtgcaacttaaagagagtggccctggtctggtggctcctggtggaagcttgtctatcacgtgtacagtatccggatttagcttgacagacagttcaatcaattgggtacgccaacctccgggtaaaggtctggagtggctcggaatgatatggggtgacggtcggatagattacgctgatgcattgaaatctaggctcagcatatctaaggactctagcaagagtcaggtttttctggagatgacgagtctccgcacggacgacaccgcaacctactattgtgccagggatggctattttccctatgcgatggacttctggggccaaggaacgagtgtcaccgtcagttcaggaggtggcggctctggaggtggaggcagtgggggcggtggctctgacattgtgttgacccaaagcccagcctccttggcggttagccttggacagagggcaacaatctcttgtcgggccagcgagtccgtggacagctacggacagtcatatatgcattggtaccagcagaaggcgggtcaaccccccaagctcctcatatacttggcttctaatttggagagtggggttcctgcgagattttccggatctggttctaggactgacttcacgttgacgatagatcccgtacaggcagaagacgcagcaacctactactgccaacaaaatgcggaggactcaagaacatttgggggcggtaccaagcttgaaattaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtggctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatc

SEQ ID NO: 17 - protein, 832 aa, Artificial Sequence
  Description of Artificial Sequence Synthetic polypeptide
MDWIWRILFLVGAATGAHSEVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSSGGGGSGGGGSGGGGSDIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIKDLEPKSCDKTHTCPPCPDPKFWVLVVVGGVLACYSLLVTVAFIIFWVAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 18 - DNA, 2499 bp, Artificial Sequence
  Description of Artificial Sequence Synthetic polynucleotide
atggattggatatggcgaatcttgttcctcgtcggtgccgcgaccggcgctcactctgaggtgcaacttaaagagagtggccctggtctggtggctcctggtggaagcttgtctatcacgtgtacagtatccggatttagcttgacagacagttcaatcaattgggtacgccaacctccgggtaaaggtctggagtggctcggaatgatatggggtgacggtcggatagattacgctgatgcattgaaatctaggctcagcatatctaaggactctagcaagagtcaggtttttctggagatgacgagtctccgcacggacgacaccgcaacctactattgtgccagggatggctattttccctatgcgatggacttctggggccaaggaacgagtgtcaccgtcagttcaggaggtggcggctctggaggtggaggcagtgggggcggtggctctgacattgtgttgacccaaagcccagcctccttggcggttagccttggacagagggcaacaatctcttgtcgggccagcgagtccgtggacagctacggacagtcatatatgcattggtaccagcagaaggcgggtcaaccccccaagctcctcatatacttggcttctaatttggagagtggggttcctgcgagattttccggatctggttctaggactgacttcacgttgacgatagatcccgtacaggcagaagacgcagcaacctactactgccaacaaaatgcggaggactcaagaacatttgggggcggtaccaagcttgaaattaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtggctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatcgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 19 - protein, 23 aa, Artificial Sequence
  Description of Artificial Sequence Synthetic peptide
MQIPQAPWPVVWAVLQLGWRPGW

SEQ ID NO: 20 - DNA, 69 bp, Artificial Sequence
  Description of Artificial Sequence Synthetic oligonucleotide
atgcaaataccgcaggctccgtggccagtagtatgggcagtcctccagcttggctggagacctggttgg

SEQ ID NO: 21 - protein, 134 aa, Artificial Sequence
  Description of Artificial Sequence Synthetic polypeptide
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHGS

SEQ ID NO: 22 - DNA, 402 bp, Artificial Sequence
  Description of Artificial Sequence Synthetic polynucleotide
ttcttggacagtcctgatcgaccttggaatcctccaacattctcccccgctttgctggttgtgacggaaggcgacaatgctacatttacctgcagtttctctaataccagcgaatcttttgtactgaattggtaccgcatgagccctagcaaccagaccgacaaattggcggcgtttccggaagatcgaagccaaccaggacaagattgccgatttagagttactcaactgcccaacggacgcgactttcatatgtcagtagtacgcgcgaggaggaatgatagtggaacatatctgtgtggagccattagcctggcccctaaagcgcaaattaaagaatctctgcgggcagagctgcgagttacagagcgcagagcagaggtcccaaccgcacacggatcc

SEQ ID NO: 23 - protein, 12 aa, Artificial Sequence
  Description of Artificial Sequence Synthetic peptide
CPSPLFPGPSKP

SEQ ID NO: 24 - DNA, 36 bp, Artificial Sequence
  Description of Artificial Sequence Synthetic oligonucleotide
tgcccaagtcctttgtttccgggtcctagcaagccg

SEQ ID NO: 25 - DNA, 81 bp, Artificial Sequence
  Description of Artificial Sequence Synthetic oligonucleotide
ttctgggtcctcgttgtcgtaggaggagtgcttgcgtgctatagtctccttgtaacggttgctttcattatcttttgggta

SEQ ID NO: 26 - DNA, 513 bp, Artificial Sequence
  Description of Artificial Sequence Synthetic polynucleotide
gcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatc

SEQ ID NO: 27 - protein, 369 aa, Artificial Sequence
  Description of Artificial Sequence Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHGSCPSPLFPGPSKPFWVLVVVGGVLACYSLLVTVAFIIFWVLEAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 28 - DNA, 1110 bp, Artificial Sequence
  Description of Artificial Sequence Synthetic polynucleotide
atgcaaataccgcaggctccgtggccagtagtatgggcagtcctccagcttggctggagacctggttggttcttggacagtcctgatcgaccttggaatcctccaacattctcccccgctttgctggttgtgacggaaggcgacaatgctacatttacctgcagtttctctaataccagcgaatcttttgtactgaattggtaccgcatgagccctagcaaccagaccgacaaattggcggcgtttccggaagatcgaagccaaccaggacaagattgccgatttagagttactcaactgcccaacggacgcgactttcatatgtcagtagtacgcgcgaggaggaatgatagtggaacatatctgtgtggagccattagcctggcccctaaagcgcaaattaaagaatctctgcgggcagagctgcgagttacagagcgcagagcagaggtcccaaccgcacacggatcctgcccaagtcctttgtttccgggtcctagcaagccgttctgggtcctcgttgtcgtaggaggagtgcttgcgtgctatagtctccttgtaacggttgctttcattatcttttgggtactcgaggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatctaa

SEQ ID NO: 29 - DNA, 69 bp, Artificial Sequence
  Description of Artificial Sequence Synthetic oligonucleotide
atgcagataccgcaggccccctggccagtagtctgggcggttctccaacttggatggcgacctggttgg

SEQ ID NO: 30 - protein, 134 aa, Artificial Sequence
  Description of Artificial Sequence Synthetic polypeptide
FLDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHGS

SEQ ID NO: 31 - DNA, 402 bp, Artificial Sequence
  Description of Artificial Sequence Synthetic polynucleotide
ttcctcgattcacccgatcgaccctggaacccaccaacattctctccggcgttgttggtggtgactgagggcgacaatgcgacctttacgtgcagcttctcaaatacatctgaaagtttccatgttgtctggcacagagagtccccttctgggcagactgatacgctcgccgctttccctgaggataggagccagcctggccaagactgccggttccgcgttacacagctcccgaatggccgcgactttcacatgagcgtcgttcgagctcggcgaaacgatagtggtacatacgtctgtggtgtcatctcccttgccccaaaaatccagatcaaggaatcccttcgcgccgaacttagagtgactgaacgacgggctgaggtgccaactgcccacggatcc

SEQ ID NO: 32 - protein, 369 aa, Artificial Sequence
  Description of Artificial Sequence Synthetic polypeptide
MQIPQAPWPVVWAVLQLGWRPGWFLDSPDRPWNPPTFSPALLVVTEGDNATFTCSFSNTSESFHVVWHRESPSGQTDTLAAFPEDRSQPGQDCRFRVTQLPNGRDFHMSVVRARRNDSGTYVCGVISLAPKIQIKESLRAELRVTERRAEVPTAHGSCPSPLFPGPSKPFWVLVVVGGVLACYSLLVTVAFIIFWVLEAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 33 - DNA, 1110 bp, Artificial Sequence
  Description of Artificial Sequence Synthetic polynucleotide
atgcagataccgcaggccccctggccagtagtctgggcggttctccaacttggatggcgacctggttggttcctcgattcacccgatcgaccctggaacccaccaacattctctccggcgttgttggtggtgactgagggcgacaatgcgacctttacgtgcagcttctcaaatacatctgaaagtttccatgttgtctggcacagagagtccccttctgggcagactgatacgctcgccgctttccctgaggataggagccagcctggccaagactgccggttccgcgttacacagctcccgaatggccgcgactttcacatgagcgtcgttcgagctcggcgaaacgatagtggtacatacgtctgtggtgtcatctcccttgccccaaaaatccagatcaaggaatcccttcgcgccgaacttagagtgactgaacgacgggctgaggtgccaactgcccacggatcctgcccaagtcctttgtttccgggtcctagcaagccgttctgggtcctcgttgtcgtaggaggagtgcttgcgtgctatagtctccttgtaacggttgctttcattatcttttgggtactcgaggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatctaa

SEQ ID NO: 34 - protein, 21 aa, Artificial Sequence
  Description of Artificial Sequence Synthetic peptide
GSGEGRGSLLTCGDVEENPGP

SEQ ID NO: 35 - protein, 63 aa, Artificial Sequence
  Description of Artificial Sequence Synthetic polypeptide
GSGSRVTELLYRMKRAETYCPRPLLAIHPTEARHKQKIVAPVKQLLNFDLLKLAGDVESNPGP

SEQ ID NO: 36 - protein, 19 aa, Amphimedon sp.
LLCFLLLLLSGDVELNPGP

SEQ ID NO: 37 - protein, 20 aa, Amphimedon sp.
HHFMFLLLLLAGDIELNPGP

SEQ ID NO: 38 - protein, 20 aa, Saccoglossus kowalevskii
WFLVLLSFILSGDIEVNPGP

SEQ ID NO: 39 - protein, 20 aa, Branchiostoma floridae
KNCAMYMLLLSGDVETNPGP

SEQ ID NO: 40 - protein, 20 aa, Branchiostoma floridae
MVISQLMLKLAGDVEENPGP

SEQ ID NO: 41 - protein, 22 aa, Artificial Sequence
  Description of Artificial Sequence Synthetic peptide
GSGATNFSLLKQAGDVEENPGP

SEQ ID NO: 42 - protein, 23 aa, Artificial Sequence
  Description of Artificial Sequence Synthetic peptide
GSGQCTNYALLKLAGDVESNPGP

SEQ ID NO: 43 - protein, 8 aa, Artificial Sequence
  Description of Artificial Sequence Synthetic peptide
  MOD_RES (2)..(2): Any amino acid
  MOD_RES (4)..(4): Any amino acid
DXEXNPGP

SEQ ID NO: 44 - protein, 296 aa, Artificial Sequence
  Description of Artificial Sequence Synthetic polypeptide
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 45 - DNA, 888 bp, Artificial Sequence
  Description of Artificial Sequence Synthetic polynucleotide
atggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatccagtttgtgcaggaaatgattcgacagcttgaacagaccaattacaggctgaaactgtgcgtgtcagaccgagatgtgctgccaggaacttgcgtatggtctatcgcttccgaactgatcgagaaacgatgccggcgcatggtagtcgtcgtaagtgacgattatttgcaatccaaagaatgtgattttcagactaaattcgcattgagtctgtccccaggtgcccatcaaaaacggctcatacccattaagtataaggcgatgaagaaagaatttccctccatcctcagattcataactgtctgtgactataccaatccttgcactaagagttggttttggacccgattggcaaaagccctttccctgccg